SEQUENCE LISTING (30 sequences)

SEQ ID NO: 1 - protein, 124 aa, synthetic construct
  REGION 1..124: note = Synthetic
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 2 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 3 - protein, 124 aa, synthetic construct
  REGION 1..124: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 4 - protein, 124 aa, synthetic construct
  REGION 1..124: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 5 - protein, 8 aa, synthetic construct
  REGION 1..8: note = Synthetic
GFTFSSYG

SEQ ID NO: 6 - protein, 8 aa, synthetic construct
  REGION 1..8: note = Synthetic
ISYAGNNK

SEQ ID NO: 7 - protein, 17 aa, synthetic construct
  REGION 1..17: note = Synthetic
AKDSYYDFLTDPDVLDI

SEQ ID NO: 8 - protein, 6 aa, synthetic construct
  REGION 1..6: note = Synthetic
QSISSY

SEQ ID NO: 9 - skipped

SEQ ID NO: 10 - protein, 10 aa, synthetic construct
  REGION 1..10: note = Synthetic
QQSYSTPPIT

SEQ ID NO: 11 - protein, 8 aa, synthetic construct
  REGION 1..8: note = Synthetic
GFTFDDYS

SEQ ID NO: 12 - protein, 8 aa, synthetic construct
  REGION 1..8: note = Synthetic
ISWNSGSI

SEQ ID NO: 13 - protein, 17 aa, synthetic construct
  REGION 1..17: note = Synthetic
AKDGSGYGKFYYYGMDV

SEQ ID NO: 14 - protein, 8 aa, synthetic construct
  REGION 1..8: note = Synthetic
GFTFDDYS

SEQ ID NO: 15 - protein, 8 aa, synthetic construct
  REGION 1..8: note = Synthetic
ISWNSGSI

SEQ ID NO: 16 - protein, 17 aa, synthetic construct
  REGION 1..17: note = Synthetic
AKYGSGYGKFYYYGMDV

SEQ ID NO: 17 - protein, 450 aa, synthetic construct
  REGION 1..450: note = Synthetic
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 450 aa, synthetic construct
  REGION 1..450: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 20 - protein, 450 aa, synthetic construct
  REGION 1..450: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 21 - protein, 117 aa, synthetic construct [sequence omitted]

SEQ ID NO: 22 - protein, 107 aa, synthetic construct [sequence omitted]

SEQ ID NO: 23 - protein, 8 aa, synthetic construct
  REGION 1..8: note = Synthetic
GFTFSNFG

SEQ ID NO: 24 - protein, 8 aa, synthetic construct
  REGION 1..8: note = Synthetic
ISGGGRDT

SEQ ID NO: 25 - protein, 10 aa, synthetic construct
  REGION 1..10: note = Synthetic
VKWGNIYFDY

SEQ ID NO: 26 - protein, 6 aa, synthetic construct
  REGION 1..6: note = Synthetic
LSINTF

SEQ ID NO: 27 - skipped

SEQ ID NO: 28 - protein, 9 aa, synthetic construct
  REGION 1..9: note = Synthetic
QQSSNTPFT

SEQ ID NO: 29 - protein, 444 aa, synthetic construct
  REGION 1..444: note = Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 214 aa, synthetic construct
  REGION 1..214: note = Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0304714 A1
(43) Pub. Date: Oct. 2, 2025

(54) Methods of Treating Metastatic Castration-Resistant Prostate Cancer with Bispecific Anti-PSMA x Anti-CD3 Antibodies Alone or in Combination with Anti-PD-1 Antibodies

(71) Applicants: Regeneron Pharmaceuticals, Inc., Tarrytown, NY (US); Regeneron Pharmaceuticals, Inc.

(72) Inventors: Elizabeth Miller, Pelham, NY (US); Sabina Sandigursky, Long Island City, NY (US); Israel Lowy, Dobbs Ferry, NY (US); Frank Seebach, Montclair, NJ (US)

(21) Appl. No.: 18/865,527

(22) PCT Filed: May 15, 2023
(86) PCT No.: PCT/US2023/022243

§ 371 Date: Nov. 13, 2024

Related U.S. Application Data
(60) Provisional application No. 63/342,548, filed on May 16, 2022.

(51) Int. Cl. C07K 16/30; A61K 39/00; A61P 35/00; C07K 16/28

(52) U.S. Cl. CPC C07K 16/3069; A61P 35/00; C07K 16/2809; C07K 16/2818; A61K 2039/507; A61K 2039/545

(57) ABSTRACT

The present disclosure provides methods for treating, reducing the severity, or inhibiting the growth of cancer (e.g., prostate cancer or metastatic castration-resistant prostate cancer). The methods of the present disclosure comprise administering to a subject in need thereof a therapeutically effective amount of a bispecific antibody or antigen-binding fragment thereof that specifically binds prostate-specific membrane antigen (PSMA) and CD3 alone or in combination with an antibody or antigen-binding fragment thereof that specifically binds to programmed death 1 (PD-1) receptor.

### REFERENCE TO A SEQUENCE LISTING

This application incorporates by reference a computer readable Sequence Listing in ST.26 XML format, titled 11050WO01_Sequence, created on May 15, 2023 and containing 38,992 bytes.

### FIELD OF THE INVENTION

The present invention relates to methods for treating cancer comprising administering to a subject in need thereof a therapeutically effective amount of a bispecific antibody that specifically binds to prostate-specific membrane antigen (PSMA) and CD3 alone, or in combination with an antibody that specifically binds to programmed death 1 (PD-1) receptor.

### BACKGROUND

Prostate-specific membrane antigen (PSMA), also known as FOLH1, glutamate carboxypeptidase II (GCPII), N-acetyl-L-aspartyl-L-glutamate peptidase I (NAALADase 1), or N-acetyl-aspartylglutamate (NAAG) peptidase, is a homodimeric, enzymatic type II transmembrane protein encoded by the folate hydrolase 1 (FOLH1) gene. PSMA is an integral, non-shed membrane glycoprotein highly expressed on malignant prostate tissue and is a cell-surface marker for prostate cancer, but shows limited expression on normal tissue. Its expression is maintained in castrate-resistant prostate cancer, a condition with poor outcome and limited treatment options. Methods for treating prostate cancer by targeting PSMA have been investigated. For example, Yttrium-90 capromab is a radiotherapeutic comprising a monoclonal antibody to an intracellular epitope of PSMA. In another example, J591, a monoclonal antibody to an extracellular epitope of PSMA, is part of the radiotherapeutic Lutetium-177 J591 and in MLN2704, in which maytansinoid 1 (DM1, an antimicrotubule agent) is conjugated to J591. These therapies have been associated with toxicity. PSMA is also expressed within the neovasculature of other tumors such as bladder, renal, gastric, and colorectal carcinomas.

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

Programmed death-1 (PD-1) receptor signaling in the tumor microenvironment plays a key role in allowing tumor cells to escape immune surveillance by the host immune system. Blockade of the PD-1 signaling pathway has demonstrated clinical activity in patients with multiple tumor types, and antibody therapeutics that block PD-1 (e.g., nivolumab and pembrolizumab) have been approved for the treatment of metastatic melanoma and metastatic squamous non-small cell lung cancer. Recent data has demonstrated the clinical activity of PD-1 blockade in patients with aggressive NHL and Hodgkin's lymphoma (Lesokhin, et al. 2014, Abstract 291, 56th ASH Annual Meeting and Exposition, San Francisco, Calif.; Ansell et al. 2015, N. Engl. J. Med. 372(4):311-9).

Prostate cancer is the leading cause of new cancer diagnoses and the second most common cause of cancer-related death in men in the United States. There were 1.3 million new cases of prostate cancer and 358,989 deaths estimated worldwide in 2018. Therapies blocking androgen related pathways have been the standard for decades in treating prostate cancers. However, patients progress on androgen depletion and/or surgical castration and develop castration resistant prostate cancer. Prognosis is especially poor for men with metastatic castration resistant prostate cancer (mCRPC). Currently, metastatic prostate cancers remain incurable and improvement in long-term survival remains a high unmet need.

### BRIEF SUMMARY OF THE INVENTION

According to certain embodiments, the present disclosure provides methods for treating, ameliorating at least one symptom or indication, or inhibiting the growth of a PSMA-expressing cancer in a subject. The methods according to this aspect of the disclosure comprise administering a therapeutically effective amount of a bispecific antibody that specifically binds to prostate specific membrane antigen (PSMA) and CD3 alone, or in combination with an antibody or antigen-binding fragment thereof that specifically binds to programmed death 1 (PD-1) to a subject in need thereof.

In certain embodiments of the present disclosure, methods are provided for treating, ameliorating at least one symptom or indication, or inhibiting the growth of a PSMA-expressing cancer in a subject. In certain embodiments of the present disclosure, methods are provided for delaying the growth of a tumor or preventing tumor recurrence. The methods, according to this and other aspects of the disclosure, comprise sequentially administering one or more doses of a therapeutically effective amount of a bispecific anti-PSMA×anti-CD3 antibody alone or in combination with one or more doses of a therapeutically effective amount of an anti-PD-1 antibody or antigen-binding fragment thereof to a subject in need thereof.

In one aspect, the present disclosure provides a method of treating a PSMA-expressing cancer in a subject in need thereof, comprising administering to the subject a bispecific antibody comprising a first antigen-binding domain that specifically binds prostate specific membrane antigen (PSMA) on a target tumor cell, and a second antigen-binding domain that specifically binds human CD3 on a T cell, wherein the bispecific antibody is administered to the subject at a dose of at least 0.03 mg.

In some embodiments, the PSMA-expressing cancer is prostate cancer. In some cases, the PSMA-expressing cancer is metastatic prostate cancer. In some cases, the PSMA-expressing cancer is castration-resistant prostate cancer.

In some embodiments, the subject has received at least two prior therapies for metastatic and/or castration-resistant prostate cancer. In some cases, the subject has received at least one anti-androgen therapy. In some embodiments, the anti-androgen therapy is selected from abiraterone, enzalutamide, apalutamide, or darolutamide.

In some embodiments, the subject has histologically or cytologically confirmed adenocarcinoma of the prostate without pure small cell carcinoma.

In some embodiments, the subject has metastatic castration-resistant prostate cancer with a prostate specific antigen (PSA) value of 4 ng/ml prior to treatment with the bispecific antibody.

In some cases, the subject's cancer has progressed within a six month period prior to treatment with the bispecific antibody, wherein cancer progression is determined by: (a) a rising PSA level confirmed with an interval of ≥1 week between each assessment; (b) radiographic disease progression in soft tissue with or without a rise in PSA; and/or (c) radiographic disease progression in bone with an appearance of two or more bone lesions on bone scan with or without a rise in PSA.

In some embodiments, the subject has had an orchiectomy. In some embodiments, the subject is receiving luteinizing hormone-releasing hormone (LHRH) agonist or antagonist therapy, and has a serum testosterone level of <50 ng/ml prior to treatment with the bispecific antibody.

In any of the various embodiments discussed above or herein, the first antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 1; and (b) three light chain complementarity determining regions (LCDR1, LCDR2 and LCDR3) contained within a light chain variable region (LCVR) comprising the amino acid sequence of SEQ ID NO: 2. In some cases, the first antigen-binding domain comprises a HCDR1 comprising the amino acid sequence of SEQ ID NO: 5, a HCDR2 comprising the amino acid sequence of SEQ ID NO: 6, and a HCDR3 comprising the amino acid sequence of SEQ ID NO: 7. In some cases, the first antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10. In some embodiments, the first antigen-binding domain comprises a HCVR comprising the amino acid sequence of SEQ ID NO: 1, and a LCVR comprising the amino acid sequence of SEQ ID NO: 2.

In any of the various embodiments discussed above or herein, the second antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 4; and (b) three light chain complementarity determining regions (LCDR1, LCDR2 and LCDR3) contained within a light chain variable region (LCVR) comprising the amino acid sequence of SEQ ID NO: 2. In some cases, the second antigen-binding domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 16. In some cases, the second antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10. In some embodiments, the second antigen-binding domain comprises a HCVR comprising the amino acid sequence of SEQ ID NO: 4, and a LCVR comprising the amino acid sequence of SEQ ID NO: 2.

In any of the various embodiments discussed above or herein, the second antigen-binding domain of the bispecific antibody comprises: (a) three heavy chain complementarity determining regions (HCDR1, HCDR2 and HCDR3) contained within a heavy chain variable region (HCVR) comprising the amino acid sequence of SEQ ID NO: 3; and (b) three light chain complementarity determining regions (LCDR1, LCDR2 and LCDR3) contained within a light chain variable region (LCVR) comprising the amino acid sequence of SEQ ID NO: 2. In some cases, the second antigen-binding domain comprises a HCDR1 comprising the amino acid sequence of SEQ ID NO: 11, a HCDR2 comprising the amino acid sequence of SEQ ID NO: 12, and a HCDR3 comprising the amino acid sequence of SEQ ID NO: 13. In some cases, the second antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10. In some embodiments, the second antigen-binding domain comprises a HCVR comprising the amino acid sequence of SEQ ID NO: 3, and a LCVR comprising the amino acid sequence of SEQ ID NO: 2.

In any of the various embodiments discussed above or herein, the bispecific antibody may comprise a human IgG heavy chain constant region. In some cases, the human IgG heavy chain constant region is isotype IgG1. In some cases, the human IgG heavy chain constant region is isotype IgG4.

In any of the various embodiments discussed above or herein, the bispecific antibody may comprise a chimeric hinge that reduces Fcγ receptor binding relative to a wild-type hinge of the same isotype.

In any of the various embodiments discussed above or herein, the first heavy chain of the bispecific antibody or the second heavy chain of the bispecific antibody, but not both, may comprise a CH3 domain comprising a H435R (EU numbering) modification and a Y436F (EU numbering) modification.

In any of the various embodiments discussed above or herein, the bispecific antibody comprises a first heavy chain comprising the amino acid sequence of SEQ ID NO: 17.

In any of the various embodiments discussed above or herein (except where the sequences are mutually exclusive), the bispecific antibody comprises a second heavy chain comprising the amino acid sequence of SEQ ID NO: 20.

In any of the various embodiments discussed above or herein (except where the sequences are mutually exclusive), the bispecific antibody comprises a second heavy chain comprising the amino acid sequence of SEQ ID NO: 19.

In any of the various embodiments discussed above or herein, the bispecific antibody comprises a light chain comprising the amino acid sequence of SEQ ID NO: 18.

In any of the various embodiments discussed above or herein (except where the sequences are mutually exclusive), the bispecific antibody comprises a first heavy chain comprising the amino acid sequence of SEQ ID NO: 17, a second heavy chain comprising the amino acid sequence of SEQ ID NO: 20, and a common light chain comprising the amino acid sequence of SEQ ID NO: 18.

In any of the various embodiments discussed above or herein (except where the sequences are mutually exclusive), the bispecific antibody comprises a first heavy chain comprising the amino acid sequence of SEQ ID NO: 17, a second heavy chain comprising the amino acid sequence of SEQ ID NO: 19, and a common light chain comprising the amino acid sequence of SEQ ID NO: 18.

In any of the various embodiments discussed above or herein, the method may further comprise administering a second therapeutic agent or therapeutic regimen. In some embodiments, the second therapeutic agent or therapeutic regimen comprises an anti-PD-1 antibody or antigen-binding fragment thereof.

In some embodiments, the anti-PD-1 antibody or antigen-binding fragment comprises: (a) three heavy chain complementarity determining regions (HCDR1, HCDR2 and HCDR3) contained within a heavy chain variable region (HCVR) comprising the amino acid sequence of SEQ ID NO: 21; and (b) three light chain complementarity determining regions (LCDR1, LCDR2 and LCDR3) contained within a light chain variable region (LCVR) comprising the amino acid sequence of SEQ ID NO: 22. In some cases, the anti-PD-1 antibody or antigen-binding fragment comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25. In some cases, the anti-PD-1 antibody or antigen-binding fragment comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28. In some embodiments, the anti-PD-1 antibody or antigen-binding fragment comprises a HCVR comprising the amino acid sequence of SEQ ID NO: 21, and a LCVR comprising the amino acid sequence of SEQ ID NO: 22. In some embodiments, the anti-PD-1 antibody or antigen-binding fragment is an anti-PD-1 antibody comprising a heavy chain comprising the amino acid sequence of SEQ ID NO: 29 and a light chain comprising the amino acid sequence of SEQ ID NO: 30.

In any of the various embodiments discussed above or herein, the bispecific antibody may be administered to the subject at a dose of from 0.01 mg to 1000 mg weekly. In some cases, the bispecific antibody is administered to the subject at a dose of from 0.03 mg to 30 mg weekly. In some cases, the bispecific antibody is administered to the subject at a dose of from 3 mg to 900 mg weekly. In some cases, the bispecific antibody is administered to the subject at a dose of from 30 mg to 900 mg weekly. In some cases, the bispecific antibody is administered to the subject at a dose of from 300 mg to 900 mg weekly.

In any of the various embodiments discussed above or herein, the bispecific antibody may be administered to the subject at a dose of from 0.01 mg to 1000 mg once every three weeks. In some cases, the bispecific antibody is administered to the subject at a dose of from 0.03 mg to 30 mg once every three weeks. In some cases, the bispecific antibody is administered to the subject at a dose of from 3 mg to 900 mg once every three weeks. In some cases, the bispecific antibody is administered to the subject at a dose of from 30 mg to 900 mg once every three weeks. In some cases, the bispecific antibody is administered to the subject at a dose of from 300 mg to 900 mg once every three weeks.

In any of the various embodiments discussed above or herein, the anti-PD-1 antibody may be administered to the subject at a dose of from 300 to 400 mg once every three weeks. In some cases, the anti-PD-1 antibody is administered to the subject at a dose of 350 mg once every three weeks.

In any of the various embodiments discussed above or herein, the subject has stable disease, a partial response, or a complete response following administration of the bispecific antibody for at least one week at a dose of from 0.03 mg to 900 mg.

In any of the various embodiments discussed above or herein, the subject may be subjected to radiographic imaging prior to and/or following administration of one or more doses of the bispecific antibody. In some cases, the radiographic imaging comprises a Fluorine F18 DCFPyL PET/CT scan.

The present disclosure also encompasses the use of the bispecific antibodies and/or the anti-PD-1 antibodies in the manufacture of a medicament for treating a PSMA-expressing cancer as set forth in any of the embodiments of the methods discussed above or herein. The present disclosure also encompasses bispecific antibodies and/or anti-PD-1 antibodies for use in any of the embodiments of the methods discussed above or herein. The present disclosure also encompasses pharmaceutical compositions comprising the bispecific antibodies and/or anti-PD-1 antibodies for use in any of the embodiments of the methods discussed above or herein.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments of the present invention will become apparent from a review of the ensuing detailed description.

### BRIEF DESCRIPTION OF THE FIGURES

FIG. 1 illustrates an embodiment of the patient-level study schema for Module 1 (QW dosing), as discussed in Example 6.

FIG. 2 illustrates an embodiment of the patient-level study schema for Module 1 (Q3W dosing), as discussed in Example 6.

FIG. 3 illustrates an embodiment of the patient-level study schema for Module 2, as discussed in Example 6.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Methods for Treating or Inhibiting the Growth of Cancers

The present disclosure includes methods for treating, ameliorating or reducing the severity of at least one symptom or indication, or inhibiting the growth of a cancer (e.g., metastatic castration-resistant prostate cancer) in a subject. The methods according to this aspect of the disclosure comprise administering a therapeutically effective amount of a bispecific antibody against PSMA and CD3 alone, or in combination with a therapeutically effective amount of an antibody or antigen-binding fragment thereof that specifically binds PD-1 to a subject in need thereof. As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, including a prostate cancer (e.g., metastatic castration-resistant prostate cancer) and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, unexplained pain, unexplained weight loss, fever, night sweats, persistent fatigue, loss of appetite, and/or enlargement of spleen. The expression includes subjects with primary or established prostate tumors. In specific embodiments, the expression includes human subjects that have and need treatment for prostate cancer or another tumor expressing PSMA. In other specific embodiments, the expression includes subjects with PSMA+ tumors (e.g., a tumor with PSMA expression as determined by flow cytometry). In certain embodiments, the expression “a subject in need thereof” includes patients with a prostate cancer that is resistant to or refractory to or is inadequately controlled by prior therapy (e.g., treatment with a conventional anti-cancer agent, including anti-androgen therapy). For example, the expression includes subjects who have been treated with chemotherapy, or anti-androgen therapy such as, for example, abiraterone, enzalutamide, apalutamide, or darolutamide. The expression also includes subjects with a prostate tumor for which conventional anti-cancer therapy is inadvisable, for example, due to toxic side effects. For example, the expression includes patients who have received one or more cycles of chemotherapy or other anti-cancer therapy with toxic side effects. In certain embodiments, the expression “a subject in need thereof” includes patients with a prostate tumor which has been treated but which has subsequently relapsed or metastasized. For example, patients with a prostate tumor that may have received treatment with one or more anti-cancer agents leading to tumor regression; however, subsequently have relapsed with cancer resistant to the one or more anti-cancer agents (e.g., castration-resistant prostate cancer) are treated with the methods of the present disclosure.

In certain embodiments, the methods of the present disclosure may be used to treat patients that have histologically or cytologically confirmed adenocarcinoma of the prostate without pure small cell carcinoma. In certain embodiments, the methods of the present disclosure may be used to treat patients that have metastatic castration-resistant prostate cancer with a prostate specific antigen (PSA) value of 24 ng/ml (e.g., 4 ng/ml, 4.5 ng/ml, 5 ng/ml, 5.5 ng/ml, 6 ng/ml, 6.5 ng/ml, 7 ng/ml, 7.5 ng/ml, 8 ng/ml, 8.5 ng/ml, 9 ng/ml, 9.5 ng/ml, or 10 ng/ml or more) prior to treatment with the bispecific antibody. In certain embodiments, the methods of the present disclosure may be used to treat patients with prostate cancer that has progressed within a period (e.g., 1 month, 2 months, 3 months, 4 months, 5 months, 6 months, 7 months, 8 months, 9 months, or more) prior to treatment with the bispecific antibody, wherein cancer progression is determined by, for example: (a) a rising PSA level confirmed with an interval of ≥1 week (e.g., 1 week, 2 weeks, 3 weeks, 4 weeks, or more) between each assessment; (b) radiographic disease progression in soft tissue with or without a rise in PSA; and/or (c) radiographic disease progression in bone with an appearance of two or more bone lesions on bone scan with or without a rise in PSA. In certain embodiments, the methods of the present disclosure may be used to treat patients that have had an orchiectomy. In certain embodiments, the methods of the present disclosure may be used to treat patient that have or are receiving luteinizing hormone-releasing hormone (LHRH) agonist or antagonist therapy, and have a serum testosterone level of <50 ng/ml (e.g., from 1 ng/ml to 49 ng/ml, about 45 ng/ml, about 40 ng/ml, about 35 ng/ml, about 30 ng/ml, about 25 ng/ml, about 20 ng/ml, about 15 ng/ml, about 10 ng/ml, or about 5 ng/ml) prior to treatment with the bispecific antibody.

In certain embodiments, the methods of the present disclosure are used in a subject with prostate cancer. The terms “tumor”, “cancer” and “malignancy” are interchangeably used herein. The term “prostate cancer”, as used herein, refers to tumors of the prostate, including metastatic tumors originating in the prostate.

According to certain embodiments, the present disclosure includes methods for treating, or delaying or inhibiting the growth of a tumor. In certain embodiments, the present disclosure includes methods to promote tumor regression. In certain embodiments, the present disclosure includes methods to reduce tumor cell load or to reduce tumor burden. In certain embodiments, the present disclosure includes methods to prevent tumor recurrence. The methods, according to this aspect of the disclosure, comprise administering a therapeutically effective amount of a bispecific anti-PSMA/anti-CD3 antibody alone, or in combination with an anti-PD-1 antibody to a subject in need thereof, wherein each antibody is administered to the subject in multiple doses, e.g., as part of a specific therapeutic dosing regimen. For example, the therapeutic dosing regimen may comprise administering one or more doses of an anti-PSMA×CD3 antibody to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the anti-PSMA×anti-CD3 antibody is administered once a week. In certain embodiments, the anti-PSMA×anti-CD3 antibody is administered once every three weeks. In certain embodiments, the one or more doses of anti-PD-1 antibody are administered in combination with the one or more doses of a therapeutically effective amount of a bispecific anti-PSMA/anti-CD3 antibody, wherein the one or more doses of the anti-PD-1 antibody are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the anti-PD-1 antibody is administered to the subject once every three weeks.

In certain embodiments, each dose of the anti-PSMA/anti-CD3 antibody is administered in two or more fractions, e.g., in 2-5 fractions (“split dosing”) within the given dosing period. The anti-PSMA/anti-CD3 bispecific antibody may be administered in split doses to reduce or eliminate the cytokine “spikes” induced in response to administration of the antibody. Cytokine spikes refer to the clinical symptoms of the cytokine release syndrome (“cytokine storm”) and infusion related reactions. In certain embodiments, the methods of the present disclosure comprise administering one or more doses of anti-PD-1 antibody in combination with the one or more doses of a bispecific anti-PSMA/anti-CD3 antibody to a subject in need thereof, wherein a dose of the bispecific antibody is administered as split doses, or in more than 1 fractions, e.g., as 2 fractions, as 3 fractions, as 4 fractions or as 5 fractions within the given dosing period. In certain embodiments, a dose of the bispecific antibody is split into 2 or more fractions, wherein each fraction comprises an amount of the antibody equal to the other fractions. In certain embodiments, a dose of the bispecific antibody is administered split into 2 or more fractions, wherein the fractions comprise unequal amounts of the antibody, e.g., more than or less than the first fraction.

In certain embodiments, the present disclosure includes methods to inhibit, retard or stop tumor metastasis or tumor infiltration into peripheral organs. The methods, according to this aspect, comprise administering a therapeutically effective amount of a bispecific anti-PSMA/anti-CD3 antibody alone, or in combination with an anti-PD-1 antibody to a subject in need thereof.

In specific embodiments, the present disclosure provides methods for increased anti-tumor efficacy or increased tumor inhibition. The methods, according to this aspect of the disclosure, comprise administering to a subject with prostate cancer a therapeutically effective amount of an anti-PD-1 antibody prior to administering a therapeutically effective amount of a bispecific anti-PSMA/anti-CD3 antibody, wherein the anti-PD-1 antibody may be administered about 1 day, more than 1 day, more than 2 days, more than 3 days, more than 4 days, more than 5 days, more than 6 days, more than 7 days, or more than 8 days prior to the bispecific antibody. In certain embodiments, the methods provide for increased tumor inhibition, e.g., by about 20%, more than 20%, more than 30%, more than 40% more than 50%, more than 60%, more than 70% or more than 80% as compared to a subject administered the bispecific antibody alone.

In certain embodiments, the methods of the present disclosure are used to treat a patient with a MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry). The methods, according to this aspect of the disclosure, comprise administering a bispecific anti-PSMA/anti-CD3 antibody alone, or in combination with an anti-PD-1 antibody to a subject in need thereof.

The methods of the present disclosure, according to certain embodiments, comprise administering to a subject a therapeutically effective amount of a bispecific anti-PSMA/anti-CD3 antibody alone, or in combination with an anti-PD-1 antibody and, optionally, a third therapeutic agent. The third therapeutic agent may be an agent selected from the group consisting of, e.g., radiation, chemotherapy, surgery, a cancer vaccine, an oncolytic virus, a PD-L1 inhibitor (e.g., an anti-PD-L1 antibody), a LAG3 inhibitor (e.g., an anti-LAG3 antibody), a CTLA-4 inhibitor (e.g., an anti-CTLA-4 antibody), a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGF.beta.) inhibitor, an epidermal growth factor receptor (EGFR) inhibitor, an antibody to a tumor-specific antigen, a cytotoxin, a chemotherapeutic agent, an IL-6R inhibitor, an IL-4R inhibitor, an IL-10 inhibitor, a cytokine such as IL-2, IL-7, IL-21, and IL-15, an anti-inflammatory drug such as corticosteroids, and non-steroidal anti-inflammatory drugs, and a dietary supplement such as anti-oxidants. In certain embodiments, the antibodies may be administered in combination with therapy including a chemotherapeutic agent, radiation and surgery. As used herein, the phrase “in combination with” means that the antibodies are administered to the subject at the same time as, just before, or just after administration of the third therapeutic agent. In certain embodiments, the antibodies and the third therapeutic agent are administered in separate formulations.

In any of the various embodiments discussed herein, the methods of the present disclosure may further comprise administration of a steroid (e.g., dexamethasone or an equivalent steroid), or an anti-IL-6 receptor antibody. In some cases, the anti-IL-6 receptor antibody is tocilizumab or sarilumab. In some cases, the steroid (e.g., dexamethasone) may be administered at a dose of from 1 mg to 20 mg (e.g., from 5 mg to 10 mg) IV or PO. In some cases, these agents may be administered as premedications prior to administration of the bispecific antibody (e.g., REGN4336).

In certain embodiments, the methods of the present disclosure comprise administering to a subject in need thereof a therapeutically effective amount of a bispecific anti-PSMA/anti-CD3 antibody alone, or in combination with an anti-PD-1 antibody. Where the bispecific antibody or the combination is administered, the administration of the antibodies leads to increased inhibition of tumor growth. In certain embodiments, tumor growth is inhibited by at least about 10%, about 20%, about 30%, about 40%, about 50%, about 60%, about 70% or about 80% as compared to an untreated subject or a subject administered with either antibody as monotherapy, respectively. In certain embodiments, the administration of the bispecific antibody or the combination leads to increased tumor regression, tumor shrinkage and/or disappearance. In certain embodiments, the administration of the bispecific antibody or the combination leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with either antibody as monotherapy, respectively. In certain embodiments, administration of the bispecific antibody or the combination prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months relative to an untreated subject or a subject which is administered either antibody as monotherapy, respectively. In certain embodiments, administration of the bispecific antibody or the combination increases progression-free survival or overall survival. In certain embodiments, administration of the bispecific antibody or the combination increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy, respectively. In certain embodiments, administration of the bispecific antibody or the combination to a subject with prostate cancer leads to complete disappearance of all evidence of tumor cells (“complete response”). In certain embodiments, administration of the bispecific antibody or the combination to a subject with prostate cancer leads to at least 30% or more decrease in tumor cells or tumor size (“partial response”). In certain embodiments, administration of the bispecific antibody or the combination to a subject with prostate cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses. In certain embodiments, administration of the bispecific antibody and the anti-PD-1 antibody produces a synergistic anti-tumor effect that exceeds the combined effects of the two agents when administered alone.

In certain cases, the response of a subject to therapy is categorized as a complete response (CR), a partial response (PR), progressive disease (PD), or as stable disease (SD). A CR is defined as disappearance of all target lesions, and a reduction in short axis of any pathological lymph nodes (whether target or non-target) to <10 mm (<1 cm). A PR is defined as an at least 30% decrease in the sum of the diameters of target lesions, taking as reference the baseline sum diameters. PD is defined as an at least 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). (Note: the appearance of one or more new lesions is also considered a progression). SD is defined as neither sufficient shrinkage to qualify for PR nor sufficient increase to qualify for PD, taking as reference the smallest sum diameters while on study.

In certain cases, immune-based therapy response criteria may be used to evaluate responses. Immune-based therapy response criteria differ from RECIST (Version 1.1) in that progressive disease is slightly more challenging to confirm, as it must occur in the scan immediately following an unconfirmed progressive disease scan. This difference is based upon understanding that immune therapies may cause pseudoprogression based on inflammation up to and including development of new lesions. Hence, two scans no less than 4 weeks apart must agree that disease is progressing for confirmed progressive disease. The criteria used to evaluate target lesions and non-target lesions are discussed below.

### Evaluation of Target Lesions

Immune Complete Response (iCR): Disappearance of all target lesions. Any pathological lymph nodes (whether target or non-target) must have reduction in short axis to <10 mm (<1 cm)
Immune Partial Response (iPR): At least a 30% decrease in the sum of the diameters of target lesions taking as reference the baseline sum diameters
Immune Unconfirmed Progressive Disease (iUPD): At least a 20% increase in the sum of the diameters of target lesions taking as reference the smallest sum on study (this includes the baseline sum if that is the smallest on study) that are new since the last imaging done. In addition to the relative increase of 20%, the sum must also demonstrate an absolute increase of at least 5 mm (0.5 cm)
Immune Confirmed Progressive Disease (iCPD): Progression is confirmed in the target lesion category if the next imaging assessment after iUPD (4 to 8 weeks later) confirms a further increase in sum or measures of target disease from iUPD, with an increase of at least 5 mm.
Immune Stable Disease (iSD): Neither sufficient shrinkage to qualify for iPR nor sufficient increase to qualify for iUPD or iCPD, taking as reference the smallest sum diameters while on study and including the measurements of new lesions

### Evaluation of Non-Target Lesions

Immune Complete Response (iCR): Disappearance of all non-target lesions and normalization of tumor marker level. All lymph nodes must be non-pathological in size (<10 mm [<1 cm] short axis). If tumor markers are initially above the upper normal limit, they must normalize for a patient to be considered in complete clinical response
Non-iCR/Non-iUPD or -iCPD: Persistence of one or more non-target lesion(s) and/or maintenance of tumor marker level above the normal limits
Immune Unconfirmed Progressive Disease (iUPD): Unequivocal progression of existing non-target lesions without an iUPD on immediate prior scan. Unequivocal progression should not normally trump target lesion status. It must be representative of overall disease status change, not a single lesion increase.
Immune Confirmed Progressive Disease (iCPD): Unequivocal progression of existing non-target lesions with an iUPD on immediate prior scan. Progressive disease in the non-target lesion category is confirmed if subsequent imaging, done 4 to 8 weeks after iUPD, shows a further increase from iUPD. Unequivocal progression should not normally trump target lesion status. It must be representative of overall disease status change, not a single lesion increase.

In certain cases, imaging may be used to evaluate a subject's response to therapy (alone or in combination with evaluation of PSA levels). PSMA PET/CT has been shown to provide a sensitive measure of both PSMA expression and tumor burden in prostate cancer patients. With improved sensitivity and specificity over current conventional imaging modalities for tumor lesion detection, PSMA PET/CT has been shown to improve the effectiveness of tumor response assessment and treatment strategy.

Fluorine F18 DCFPyL (¹⁸F-DCFPyL) is a radiolabeled small molecule that binds to the extracellular domain of PSMA with high affinity. Data from an enzyme inhibition assay showed that DCFPyL binds competitively to PSMA expressing LNCaP cells with a Ki of 1.1 nM. ¹⁸F-DCFPyL has been tested in multiple phase 1 to 3 studies and found to be well tolerated in prostate cancer patients. ¹⁸F-DCFPyL was approved on 26 May 2021 by the FDA for PET scan of PSMA-positive lesions in men with prostate cancer with suspected metastasis who are candidates for initial definitive therapy and with suspected recurrence based on elevated serum prostate-specific antigen (PSA) level.

Following administration of ¹⁸F-DCFPyL injection, biodistribution and optimal imaging time point were determined. The radiation dose used in the studies was within limit for diagnostic radiotracers for PET. Physiologic accumulation of ¹⁸F-DCFPyL was found to correspond to the distribution of PSMA expressing organs. Accumulation in primary tumor and metastatic lesions was very high, suggesting that ¹⁸F-DCFPyL injection can be used to detect residual tumor as well as regional or distant metastases with high sensitivity and specificity. Together, these findings strongly support using ¹⁸F-DCFPyL PSMA PET/CT for assessing whole body tumor burden and the anti-tumor activity of REGN4336 alone and in combination with cemiplimab in mCRPC patients. ¹⁸F-DCFPyL at the intended dose of ˜9±1 mCi per IV injection is overall feasible and safe. Radiation dose from the ¹³F-DCFPyL PET/CT scan is about 7.4 mSv. By comparison, a typical CT scan dose of the chest, abdomen and pelvis is estimated to be˜25 mSv, one bone scan is ˜4.4 mSv and annual natural background radiation dose in the US is ˜3.1 mSv. The protocol maximum of three ¹⁸F-DCFPyL PET/CT scans within a year would result in an approximate dose of ˜22.2 mSv which is ˜45% of the recommended maximum allowed dose of 50 mSv for an adult research patient in a single year (21CRF361.1). For patients receiving potentially 4 PSMA PET scans (3 on schedule plus 1 unscheduled), the total radiation dose from those scans will be ˜29.6mSv which is ˜59% of the recommended maximum allowed annual dose for an adult research patient. Risks of ¹³F-DCFPyL including radiation exposure will be disclosed to patients in the PET imaging informed consent form.

Optional fluorodeoxyglucose (FDG) PET/CT may be used to assess metabolically active tumor burden in patients. PSMA expressing and non-expressing tumors have been detected by FDG PET/CT in prostate cancer patients. Complementary FDG and PSMA PET/CT data is expected to provide insight on tumor responses to therapy (e.g., REGN4336).

### Anti-PD-1 Antibodies and Antigen-Binding Fragments Thereof

According to certain exemplary embodiments of the present disclosure, the methods comprise administering a therapeutically effective amount of an anti-PD-1 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-PD-1 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CODR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}—C_{H}1-C_{H}2; (v) V_{H}—C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (X) V_{L}-C_{H}3; (xi) V_{L}-C_{H}1-C_{H}2; (Xii) V_{L}-C_{H}1-O_{H}2-C_{H}3; (xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for PD-1 or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab.sup.2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present disclosure may be human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present disclosure specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in U.S. Pat. No. 9,987,500. In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 21 and the light chain complementarity determining regions (LCDRs) of a light chain variable region (LCVR) comprising the amino acid sequence of SEQ ID NO: 22. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 23; the HCDR2 comprises the amino acid sequence of SEQ ID NO: 24; the HCDR3 comprises the amino acid sequence of SEQ ID NO: 25; the LCDR1 comprises the amino acid sequence of SEQ ID NO: 26; the LCDR2 comprises the amino acid sequence of SEQ ID NO: 27; and the LCDR3 comprises the amino acid sequence of SEQ ID NO: 28. In yet other embodiments, the anti-PD-1 antibody or antigen-binding fragment thereof comprises an HCVR comprising SEQ ID NO: 21 and an LCVR comprising SEQ ID NO: 22. In certain embodiments, the methods of the present disclosure comprise the use of an anti-PD-1 antibody, wherein the antibody comprises a heavy chain comprising the amino acid sequence of SEQ ID NO: 29. In some embodiments, the anti-PD-1 antibody comprises a light chain comprising the amino acid sequence of SEQ ID NO: 30. An exemplary antibody comprising a heavy chain variable region comprising the amino acid sequence of SEQ ID NO: 21 and a light chain variable region comprising the amino acid sequence of SEQ ID NO: 22 is the fully human anti-PD-1 antibody known as REGN2810 (also known as cemiplimab; LIBTAYO®). According to certain exemplary embodiments, the methods of the present disclosure comprise the use of REGN2810, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of REGN2810 when administered at the same molar dose under similar experimental conditions, either single dose or multiple dose. In the context of the disclosure, the term refers to antigen-binding proteins that bind to PD-1 which do not have clinically meaningful differences with REGN2810 in their safety, purity and/or potency.

Other anti-PD-1 antibodies that can be used in the context of the methods of the present disclosure include, e.g., the antibodies referred to and known in the art as nivolumab (U.S. Pat. No. 8,008,449), pembrolizumab (U.S. Pat. No. 8,354,509), MEDI0608 (U.S. Pat. No. 8,609,089), pidilizumab (U.S. Pat. No. 8,686,119), or any of the anti-PD-1 antibodies as set forth in U.S. Pat. Nos. 6,808,710, 7,488,802, 8,168,757, 8,354,509, 8,779,105, or 8,900,587.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Bispecific Anti-PSMA×Anti-CD3 Antibodies

According to certain exemplary embodiments of the present disclosure, the methods comprise administering a therapeutically effective amount of a bispecific antibody that specifically binds CD3 and PSMA. Such antibodies may be referred to herein as, e.g., “anti-PSMA/anti-CD3,” or “anti-PSMA×CD3” or “PSMA×CD3” bispecific antibodies, or other similar terminology.

As used herein, the expression “bispecific antibody” refers to an immunoglobulin protein comprising at least a first antigen-binding domain and a second antigen-binding domain. In the context of the present disclosure, the first antigen-binding domain specifically binds a first antigen (e.g., PSMA), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present disclosure, the multimerizing component is an Fc portion of an immunoglobulin (comprising a C_{H2}-C_{H3} domain), e.g., an Fc domain of an IgG selected from the isotypes IgG1, IgG2, IgG3, and IgG4, as well as any allotype within each isotype group.

Bispecific antibodies of the present disclosure typically comprise two multimerizing domains, e.g., two Fc domains that are each individually part of a separate antibody heavy chain.

The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antibodies of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antibody. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab₂ bispecific formats (see, e.g., Klein et al. 2012, mAbs 4:6, 1-11, and references cited therein, for a review of the foregoing formats).

In the context of bispecific antibodies of the present disclosure, Fc domains may comprise one or more amino acid changes (e.g., insertions, deletions or substitutions) as compared to the wild-type, naturally occurring version of the Fc domain. For example, the disclosure includes bispecific antibodies comprising one or more modifications in the Fc domain that results in a modified Fc domain having a modified binding interaction (e.g., enhanced or diminished) between Fc and FcRn. In one embodiment, the bispecific antibody comprises a modification in a C_{H2} or a C_{H3} region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422Iby EU) in the case of IgG1 antibodies; N44S, K52N, and V82I (IMGT; N384S, K392N, and V422Iby EU) in the case of IgG2 antibodies; and Q15R, N44S, K52N, V57M, R69K, E79Q, and V82I (by IMGT; Q355R, N384S, K392N, V397M, R409K, E419Q, and V422Iby EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antibodies set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antibodies set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains or chimeric heavy chain constant regions that can be included in any of the antibodies of the present disclosure are described in US Patent Publication No. 20140243504, which is herein incorporated in its entirety. Chimeric Fc domains and chimeric heavy chain constant regions having these general structural arrangements, and variants thereof, can have altered Fc receptor binding, which in turn affects Fc effector function.

According to certain exemplary embodiments of the present disclosure, the bispecific anti-PSMA/anti-CD3 antibody, or antigen-binding fragment thereof comprises heavy chain variable regions (A-HCVR and B-HCVR), light chain variable regions (A-LCVR and B-LCVR), and/or complementarity determining regions (CDRs) comprising any of the amino acid sequences of the bispecific anti-PSMA/anti-CD3 antibodies as set forth in US Patent Publication No. 20170051074. In certain exemplary embodiments, the bispecific anti-PSMA/anti-CD3 antibody or antigen-binding fragment thereof that can be used in the context of the methods of the present disclosure comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 2; and (b) a second antigen-binding arm comprising the heavy chain CDRs (B-HCDR1, B-HCDR2 and B-HCDR3) of a HCVR (B-HCVR) comprising an amino acid sequence of SEQ ID NO: 3 or SEQ ID NO: 4, and the light chain CDRs (B-LCDR1, B-LCDR2 and B-LCDR3) of a LCVR (B-LCVR) comprising the amino acid sequence of SEQ ID NO: 2. According to certain embodiments, the A-HCDR1 comprises the amino acid sequence of SEQ ID NO: 5; the A-HCDR2 comprises the amino acid sequence of SEQ ID NO: 6; the A-HCDR3 comprises the amino acid sequence of SEQ ID NO: 7; the A-LCDR1 comprises the amino acid sequence of SEQ ID NO: 8; the A-LCDR2 comprises the amino acid sequence of SEQ ID NO: 9; the A-LCDR3 comprises the amino acid sequence of SEQ ID NO: 10; the B-HCDR1 comprises the amino acid sequence of SEQ ID NO: 11, or SEQ ID NO: 14; the B-HCDR2 comprises the amino acid sequence of SEQ ID NO: 12, or SEQ ID NO: 15; and the B-HCDR3 comprises the amino acid sequence of SEQ ID NO: 13, or SEQ ID NO: 16; and the B-LCDR1 comprises the amino acid sequence of SEQ ID NO: 8; the B-LCDR2 comprises the amino acid sequence of SEQ ID NO: 9; the B-LCDR3 comprises the amino acid sequence of SEQ ID NO: 10. In yet other embodiments, the bispecific anti-PSMA/anti-CD3 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 2; and (b) a second antigen-binding arm comprising a HCVR (B-HCVR) comprising SEQ ID NO: 3 or SEQ ID NO: 4, and a LCVR (B-LCVR) comprising SEQ ID NO: 2. In certain exemplary embodiments, the bispecific anti-PSMA×CD3 antibody comprises a PSMA-binding arm comprising a heavy chain comprising the amino acid sequence of SEQ ID NO: 17 and a light chain comprising the amino acid sequence of SEQ ID NO: 18, and a CD3-binding arm comprising a heavy chain comprising the amino acid sequence of SEQ ID NO: 19 and a light chain comprising the amino acid sequence of SEQ ID NO: 18. In certain exemplary embodiments, the bispecific ant-PSMA×CD3 antibody comprises a PSMA-binding arm comprising a heavy chain comprising the amino acid sequence of SEQ ID NO: 17 and a light chain comprising the amino acid sequence of SEQ ID NO: 18, and a CD3-binding arm comprising a heavy chain comprising the amino acid sequence of SEQ ID NO: 20 and a light chain comprising the amino acid sequence of SEQ ID NO: 18. An exemplary bispecific anti-PSMA/anti-CD3 antibody comprising: (a) a first antigen-binding arm comprising a HCVR (A-HCVR) comprising SEQ ID NO: 1 and a LCVR (A-LCVR) comprising SEQ ID NO: 2; and (b) a second antigen-binding arm comprising a HCVR (B-HCVR) comprising SEQ ID NO: 4, and a LCVR (B-LCVR) comprising SEQ ID NO: 2 that is used in the methods of the present disclosure is REGN4336 (herein referred to as “PSMA/CD3-002”).

### Combination Therapies

The methods of the present disclosure, according to certain embodiments, comprise administering to the subject an anti-PSMA/anti-CD3 bispecific antibody in combination with an anti-PD-1 antibody. In certain embodiments, the methods of the present disclosure comprise administering the antibodies for additive or synergistic activity to treat a PSMA-expressing cancer, preferably prostate cancer. As used herein, the expression “in combination with” means that the anti-PSMA/anti-CD3 bispecific antibody is administered before, after, or concurrent with the anti-PD-1 antibody. The term “in combination with” also includes sequential or concomitant administration of anti-PD-1 antibody and a bispecific anti-PSMA/anti-CD3 antibody. For example, when administered “before” the bispecific anti-PSMA/anti-CD3 antibody, the anti-PD-1 antibody may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the bispecific anti-PSMA/anti-CD3 antibody. When administered “after” the bispecific anti-PSMA/anti-CD3 antibody, the anti-PD-1 antibody may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the bispecific anti-PSMA/anti-CD3 antibody. Administration “concurrent” with the bispecific anti-PSMA/anti-CD3 antibody means that the anti-PD-1 antibody is administered to the subject in a separate dosage form within less than 5 minutes (before, after, or at the same time) of administration of the bispecific anti-PSMA/anti-CD3 antibody, or administered to the subject as a single combined dosage formulation comprising both the anti-PD-1 antibody and the bispecific anti-PSMA/anti-CD3 antibody.

In certain embodiments, the methods of the present disclosure comprise administration of a third therapeutic agent wherein the third therapeutic agent is an anti-cancer drug. In certain embodiments, the methods of the disclosure comprise administering an anti-PD-1 antibody and an anti-PSMA/anti-CD3 bispecific antibody in combination with radiation therapy, surgery or other anti-cancer therapy to generate long-term durable anti-tumor responses and/or enhance survival of patients with a PSMA-expressing cancer.

In some embodiments, the methods of the disclosure comprise administering radiation therapy prior to, concomitantly or after administering an anti-PD-1 antibody and a bispecific anti-PSMA/anti-CD3 antibody to a cancer patient. For example, radiation therapy may be administered in one or more doses to tumor lesions after administration of one or more doses of the antibodies. In some embodiments, radiation therapy may be administered locally to a tumor lesion to enhance the local immunogenicity of a patient's tumor (adjuvinating radiation) and/or to kill tumor cells (ablative radiation) after systemic administration of an anti-PD-1 antibody and/or a bispecific anti-PSMA/anti-CD3 antibody.

### Pharmaceutical Compositions and Administration

The present disclosure includes methods which comprise administering a bispecific anti-PSMA/anti-CD3 antibody alone, or in combination with an anti-PD-1 antibody to a subject wherein the antibody or antibodies are contained within separate or a combined (single) pharmaceutical composition. The pharmaceutical compositions of the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, or by injection, and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent. The injection thus prepared is preferably filled in an appropriate ampoule.

Advantageously, the pharmaceutical compositions for use described above are prepared into dosage forms in a unit dose suited to fit a dose of the active ingredients. Such dosage forms in a unit dose include, for example, a vial or a prefilled syringe.

### Administration Regimens

The present disclosure includes methods comprising administering to a subject a bispecific anti-PSMA×CD3 antibody alone or in combination with an anti-PD-1 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

According to certain embodiments of the present disclosure, multiple doses of a bispecific anti-PSMA/anti-CD3 antibody alone, or in combination with an anti-PD-1 antibody may be administered to a subject over a defined time course. The methods according to this aspect of the disclosure comprise sequentially administering to a subject one or more doses of a bispecific anti-PSMA/anti-CD3 antibody alone, or in combination with one or more doses of an anti-PD-1 antibody. As used herein, “sequentially administering” means that each dose of the antibody is administered to the subject at a different point in time, e.g., on different days separated by a predetermined interval (e.g., hours, days, weeks or months). The present disclosure includes methods which comprise sequentially administering to the patient a single initial dose of an antibody, followed by one or more secondary doses of the antibody, and optionally followed by one or more tertiary doses of the antibody.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (anti-PD-1 antibody or bispecific antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1/2 to 14 (e.g., 1/2, 1, 11/2, 2, 21/2, 3, 31/2, 4, 41/2, 5, 51/2, 6, 61/2, 7, 71/2, 8, 81/2, 9, 91/2, 10, 101/2, 11, 111/2, 12, 121/2, 13, 131/2, 14, 141/2, or more) weeks after the immediately preceding dose. The phrase “the immediately preceding dose,” as used herein, means, in a sequence of multiple administrations, the dose of a bispecific anti-PSMA/anti-CD3 (and/or anti-PD-1 antibody) which is administered to a patient prior to the administration of the very next dose in the sequence with no intervening doses.

The methods according to this aspect of the disclosure may comprise administering to a patient any number of secondary and/or tertiary doses of bispecific anti-PSMA/anti-CD3 antibody (and/or an anti-PD-1 antibody). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1, 2 or 3 weeks (e.g., 1 week or 3 weeks) after the immediately preceding dose. Similarly, in embodiments involving multiple tertiary doses, each tertiary dose may be administered at the same frequency as the other tertiary doses. For example, each tertiary dose may be administered to the patient 1 to 4 weeks (e.g., 1 week or 3 weeks) after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of a bispecific anti-PSMA/anti-CD3 antibody (e.g., and an anti-PD-1 antibody) are administered at the beginning of a treatment regimen as “induction doses” on a more frequent basis (twice a week, once a week, once in 2 weeks, or once in 3 weeks) followed by subsequent doses (“consolidation doses” or “maintenance doses”) that are administered on the same or a less frequent basis (e.g., once in 4-12 weeks).

The present disclosure includes methods comprising sequential administration of a bispecific anti-PSMA/anti-CD3 antibody alone, or in combination with an anti-PD-1 antibody to a patient to treat prostate cancer (e.g., metastatic castration-resistant prostate cancer). In some embodiments, the present methods comprise administering one or more doses of a bispecific anti-PSMA/anti-CD3 antibody, optionally preceded by or followed by one or more doses of an anti-PD-1 antibody. In certain embodiments, the present methods comprise administering a single dose of an anti-PD-1 antibody followed by one or more doses of a bispecific anti-PSMA/anti-CD3 antibody. In some embodiments, one or more doses of about 0.1 mg/kg to about 20 mg/kg (e.g., 100 to 600 mg) of an anti-PD-1 antibody may be administered followed by one or more doses of about 0.1 mg/kg to about 20 mg/kg (e.g., 0.01 to 1000 mg) of the bispecific antibody to inhibit tumor growth and/or to prevent tumor recurrence in a subject with prostate cancer. In some embodiments, the bispecific antibody or the combination results in increased anti-tumor efficacy (e.g., greater inhibition of tumor growth, increased prevention of tumor recurrence as compared to an untreated subject or a subject administered with either antibody as monotherapy, respectively). Alternative embodiments of the disclosure pertain to concomitant administration of the anti-PD-1 antibody and the bispecific antibody, which is administered at a separate dosage at a similar or different frequency relative to the anti-PD-1 antibody. In some embodiments, the bispecific antibody is administered before, after or concurrently with the anti-PD-1 antibody. In certain embodiments, the bispecific antibody is administered as a single dosage formulation with the anti-PD-1 antibody.

### Dosage

The amount of bispecific anti-PSMA/anti-CD3 antibody, and optionally anti-PD-1 antibody, administered to a subject according to the methods of the present disclosure is, generally, a therapeutically effective amount. As used herein, the phrase “therapeutically effective amount” means an amount of antibody (anti-PD-1 antibody or bispecific anti-PSMA/anti-CD3 antibody) that results in one or more of: (a) a reduction in the severity or duration of a symptom of a cancer (e.g., prostate cancer); (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with cancer (e.g., prostate cancer); and/or (g) a reduction in the use or need for conventional anti-cancer therapy (e.g., reduced or eliminated use of chemotherapeutic or cytotoxic agents) as compared to an untreated subject or a subject administered with either antibody as monotherapy.

In the case of a bispecific anti-PSMA/anti-CD3 antibody, a therapeutically effective amount can be from about 0.01 milligrams (mg) to about 2000 mg, e.g., about 0.01 mg, about 0.03 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the bispecific anti-PSMA/anti-CD3 antibody. In certain embodiments, 0.03 mg, 0.09 mg, 0.1 mg, 0.3 mg, 0.9 mg, 1 mg, 3 mg, 9 mg, 10 mg, 30 mg, 90 mg, 100 mg, 300 mg, or 900 mg of the bispecific anti-PSMA×anti-CD3 antibody is administered (e.g., once weekly or once every three weeks) to the subject to treat a PSMA-expressing cancer or prostate cancer (e.g., metastatic and/or castration-resistant prostate cancer).

In the case of an anti-PD-1 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PD-1 antibody. In certain embodiment, 300 mg to 400 mg of the anti-PD-1 antibody is administered (e.g., once every three weeks) to the subject in combination with the bispecific antibody to treat a PSMA-expressing cancer or prostate cancer (e.g., metastatic and/or castration-resistant prostate cancer). In certain embodiments, 350 mg of an anti-PD-1 antibody is administered (e.g., once every three weeks) to the subject in combination with the bispecific antibody to treat a PSMA-expressing cancer or prostate cancer (e.g., metastatic and/or castration-resistant prostate cancer).

The amount of bispecific anti-PSMA/anti-CD3 antibody and optionally anti-PD-1 antibody contained within the individual doses may be expressed in terms of milligrams of antibody per kilogram of subject body weight (i.e., mg/kg). In certain embodiments, the bispecific anti-PSMA/anti-03 antibody, and optionally the anti-PD-1 antibody, used in the methods of the present disclosure may be administered to a subject at a dose of about 0.0001 to about 100 mg/kg of subject body weight. For example, the bispecific anti-PSMA/anti-CD3 antibody may be administered at a dose of about 0.1 mg/kg to about 20 mg/kg of a patient's body weight, and the optional anti-PD-1 antibody may be administered at dose of about 0.1 mg/kg to about 20 mg/kg of a patient's body weight.

A summary of the sequences and the corresponding SEQ ID NOs referenced herein is shown in Table 1, below.

TABLE 1
Summary of Sequences

| SEQ ID NO: | Description |
|---|---|
| 1 | Anti-PSMA Heavy Chain Variable Region |
| 2 | Anti-PSMA and Anti-CD3 Light Chain Variable Region |
| 3 | Anti-CD3-G5 Heavy Chain Variable Region |
| 4 | Anti-CD3-G20 Heavy Chain Variable Region |
| 5 | Anti-PSMA HCDR1 |
| 6 | Anti-PSMA HCDR2 |
| 7 | Anti-PSMA HCDR3 |
| 8 | Anti-PSMA and Anti-CD3 LCDR1 |
| 9 | Anti-PSMA and Anti-CD3 LCDR2 |
| 10 | Anti-PSMA and Anti-CD3 LCDR3 |
| 11 | Anti-CD3-G5 HCDR1 |
| 12 | Anti-CD3-G5 HCDR2 |
| 13 | Anti-CD3-G5 HCDR3 |
| 14 | Anti-CD3-G20 HCDR1 |
| 15 | Anti-CD3-G20 HCDR2 |
| 16 | Anti-CD3-G20 HCDR3 |
| 17 | Anti-PSMA Heavy Chain |
| 18 | Anti-PSMA and Anti-CD3 Light Chain |
| 19 | Anti-CD3-G5 Heavy Chain |
| 20 | Anti-CD3-G20 Heavy Chain |
| 21 | Anti-PD-1 Heavy Chain Variable Region |
| 22 | Anti-PD-1 Light Chain Variable Region |
| 23 | Anti-PD-1 HCDR1 |
| 24 | Anti-PD-1 HCDR2 |
| 25 | Anti-PD-1 HCDR3 |
| 26 | Anti-PD-1 LCDR1 |
| 27 | Anti-PD-1 LCDR2 |
| 28 | Anti-PD-1 LCDR3 |
| 29 | Anti-PD-1 Heavy Chain |
| 30 | Anti-PD-1 Light Chain |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind Prostate-Specific Membrane Antigen (PSMA) and CD3

The present disclosure provides bispecific antibodies that bind CD3 and Prostate-Specific Membrane Antigen (PSMA); such bispecific antibodies are also referred to herein as anti-PSMA×anti-CD3 bispecific antibodies or anti-PSMA×CD3 bispecific antibodies. The anti-PSMA portion of the anti-PSMA×anti-CD3 bispecific antibodies is useful for targeting tumor cells that express PSMA, and the anti-CD3 portion of the bispecific antibodies is useful for activating T-cells. The simultaneous binding of PSMA on a tumor cell and CD3 on a T-cell facilitates directed killing (cell lysis) of the targeted tumor cell by the activated T-cell.

Bispecific antibodies comprising an anti-PSMA-specific binding domain and an anti-CD3-specific binding domain were constructed using standard methodologies, wherein the anti-PSMA antigen binding domain and the anti-CD3 antigen binding domain each comprise different, distinct HCVRs paired with a common LCVR. The bispecific antibodies were constructed utilizing a heavy chain from an anti-CD3 antibody, a heavy chain from an anti-PSMA antibody and a common light chain.

A summary of the component parts of the antigen-binding domains of the various anti-PSMA×CD3 bispecific antibodies constructed is set forth in Table 2.

TABLE 2
Summary of Component Parts of
PSMAxCD3 Bispecific Antibodies

| | Anti-PSMA | Anti-CD3 | |
|---|---|---|---|
| | Antigen-Binding | Antigen-Binding | Common |
| Bispecific | Domain | Domain | Light Chain |
| Antibody | Heavy Chain | Heavy Chain | Variable |
| Identifier | Variable Region | Variable Region | Region |
| PSMA/CD3-001 | SEQ ID NO: 1 | CD3-VH-G5 | SEQ ID NO: 2 |
| | | (SEQ ID NO: 3) | |
| PSMA/CD3-002 | SEQ ID NO: 1 | CD3-VH-G20 | SEQ ID NO: 2 |
| | | (SEQ ID NO: 4) | |

### Example 2: Binding Affinities of Exemplified Bispecific Antibodies as Measured by FACS Analysis

In this example, the ability of the anti-PSMA×anti-CD3 bispecific antibodies described in Example 1 to bind to human PSMA expressing cell lines and to human and cynomolgus CD3-expressing cell lines via FACS was determined.

Briefly, 2×10⁵ cells/well of human CD3-expressing Jurkat, cynomolgus T, or human PSMA-specific expressing cells were incubated with a serial dilution of bispecific antibodies for 30 min at 4° C. After incubation, cells were washed and a goat F(ab′)₂ anti-human Fcγ PE labeled secondary (Jackson Immunolabs) was added to the cells for an additional 30 min. Next, cells were washed, re-suspended in cold PBS+1% BSA and analyzed via flow cytometry on a BD FACS Canto II.

For FACS analysis, cells were gated by forward scatter height vs. forward scatter area for single events selection, followed by side and forward scatters. The EC₅₀ for cell binding titration was determined using Prism software. Values were calculated using 4-parameter non-linear regression analysis.

TABLE 3
FACS Binding on CD3 and PSMA-Specific Cell lines

| Bispecific | | | Cyno T- | | |
|---|---|---|---|---|---|
| Antibody | Anti-CD3- | Jurkat | cells | B16F10.9/PSMA | 22RV1 |
| Identifier | Binding Arm | EC50 [M] | EC50 [M] | EC50 [M] | EC50 [M] |
| PSMA/CD3-001 | CD3-VH-G5 | ~1.0E−06 | NB | 1.31E−09 | NT |
| PSMA/CD3-002 | CD3-VH-G20 | 2.10E−07 | 6.14E−06 | 2.09E−09 | NT |

NB = no binding; NT = not tested

As shown in Table 2, the anti-PSMA×anti-CD3 bispecific antibodies tested demonstrated specificity of binding to human PSMA-expressing B16F10.9/hPSMA and 22RV1 cell lines via FACS. The detection limit for FACS binding is 1 μM EC50.

As shown in Table 2, the CD3 binding arms of each PSMA×CD3 bispecific antibody displayed a range of cell binding affinity to human CD3 expressing Jurkat cells (15 to 300 nM EC50 range). Importantly, the CD3 arms that showed weak-to-non detectable binding to human CD3 heterodimeric protein via surface plasmon resonance (see Table 4 below) also correlated with weak to no observable binding on Jurkat cells (i.e. CD3-VH-G5). Both tested bispecific antibodies displayed similar cell binding on respective PSMA-expressing cell lines, confirming that bispecific pairing with individual CD3 arms did not affect or diminish PSMA-specific binding.

### Example 3: Binding Affinities of Exemplified Antibodies as Measured by a Surface Plasmon Resonance Binding Assay

Binding affinities and kinetic constants of anti-PSMA×anti-CD3 bispecific antibodies to soluble heterodimeric hCD3 mFc protein were determined by surface plasmon resonance at 37° C. using an antigen-capture format (Table 4). Measurements were conducted on a Sierra Sensors MASS-1 instrument.

In the antigen-capture format, the MASS-1 high-density amine sensor surface was derivatized with a goat anti-mouse IgG2a polyclonal antibody (Southern Biotech). Soluble heterodimeric CD3 protein was captured and the respective antibodies were injected over the captured antigen.

Kinetic association (kₐ) and dissociation (k_{d}) rate constants were determined by processing and fitting the data to a 1:1 binding model using MASS-1 AnalyserR2 curve fitting software. Binding dissociation equilibrium constants (K_{D}) and dissociative half-lives (t_{1/2}) were calculated from the kinetic rate constants as: K_{D} (M)=k_{d}/kₐ; and t_{1/2} (min) (In2/(60*k_{d}).

TABLE 4
Affinities of anti-CD3 Bispecific Antibodies to Soluble Human CD3

| | Binding at 37° C./Antigen-Capture Format | | | | |
|---|---|---|---|---|---|
| | Corresponding anti- | | | | |
| Bispecific | CD3 Antigen- | | | | |
| Antibody | Binding HCVR | | | | T_{1/2} |
| Identifier | Identifier | ka (Ms⁻¹) | kd (s⁻¹) | K_{D} (M) | (min) |
| PSMA/CD3-001 | CD3-VH-G5 | NB | NB | NB | NB |
| PSMA/CD3-002 | CD3-VH-G20 | 1.73E+04 | 5.77E−03 | 3.34E−07 | 2.0 |
| Control 1 | CD3-L2K | 3.68E+05 | 2.66E−03 | 7.22E−09 | 4.3 |

As shown in Table 4, the anti-PSMA×anti-CD3 bispecific antibodies either maintained very weak binding to soluble CD3 in the surface plasmon resonance binding assay, e.g. having a K_{D} value of 334 nM, or did not exhibit any detectable binding.

### Example 4: T-Cell Activation and Tumor-Specific Cytotoxicity Exhibited by Bispecific Antibodies as Measured In Vitro

In this example, the specific killing of PSMA-expressing target cells in the presence of anti-PSMA×anti-CD3 bispecific antibodies was monitored via flow cytometry. As reported previously, the bispecific antibodies displayed a range of affinity to CD3 protein and CD3-expressing cell lines. This same pair of bispecific antibodies was tested for the ability to induce naïve human T-cells to re-direct killing toward target-expressing cells.

Briefly, PSMA-expressing (C4-2, 22Rv1 and TRAMPC2_PSMA) cell lines were labeled with 1 μM of the fluorescent tracking dye Violet Cell Tracker. After labeling, cells were plated overnight at 37° C. Separately, human PBMCs were plated in supplemented RPMI media at 1×10⁶ cells/mL and incubated overnight at 37° C. in order to enrich for lymphocytes by depleting adherent macrophages, dendritic cells, and some monocytes. The next day, target cells were co-incubated with adherent cell-depleted naïve U.S. Pat. No. PBMC (Effector/Target cell 4:1 ratio) and a serial dilution of relevant bispecific antibodies or isotype control for 48 hours at 37° C. Cells were removed from cell culture plates using an enzyme-free cell dissociation buffer, and analyzed by FACS.

For FACS analysis, cells were stained with a dead/live far red cell tracker (Invitrogen). 5×10⁵ counting beads were added to each well immediately before FACS analysis. 1×10⁴ beads were collected for each sample. For the assessment of specificity of killing, cells were gated on live Violet labeled populations. Percent of live population was recorded and used for the calculation of normalized survival.

T cell activation was assessed by incubating cells with directly conjugated antibodies to CD2 and CD69, and by reporting the percent of activated (CD69+) T cells out of total T cells (CD2+).

As the results in Table 5 show, depletion of PSMA-expressing cells was observed with anti-PSMA×anti-CD3 bispecific antibodies. The tested bispecific antibodies activated and directed human T cells to deplete the target cells with EC₅₀s in the picomolar range. Additionally, the observed target-cell lysis was associated with an up-regulation of CD69 cells on CD2+ T cells, with pM EC₅₀s.

Importantly, the results of this example demonstrate that the bispecific antibody that utilized a CD3 binding arm that displayed weak-to-non-observable binding to CD3 protein or CD3-expressing cells (i.e., CD3-VH-G5) still retained the ability to activate T-cells and exhibited potent cytotoxicity of tumor antigen-expressing cells.

TABLE 5
Cytotoxicity and T-cell activation properties
of selected PSMAxCD3 Bispecific Antibodies

| Bispecific | Anti-CD3 | C4-2 Cell | 22RV1 | TrampC2.PSMA | T cell |
|---|---|---|---|---|---|
| Antibody | Binding | depletion | Cell depletion | Cell depletion | activation |
| Identifier | Arm | EC₅₀ [M] | EC₅₀ [M] | EC₅₀ [M] | EC₅₀ [M] |
| PSMA/ | CD3-VH-G5 | 2.15E−11 | 6.31E−12 | 1.15E−11 | 1.34E−11 |
| CD3-001 | | | | | |
| PSMA/ | CD3-VH- | 1.39E−11 | 8.32E−12 | NT | 6.11E−12 |
| CD3-002 | G20 | | | | |

NT = not tested

### Example 5: Anti-PSMA/Anti-CD3 Bispecific Antibodies Display Potent Anti-Tumor Efficacy In Vivo

To determine the in vivo efficacy of exemplary anti-PSMA/anti-CD3 bispecific antibodies, studies were performed in immunocompromised mice bearing human prostate cancer xenografts. Additional studies were also carried out in immunocompetent mice bearing mouse prostate cancer xenografts engineered to express human PSMA.

### Efficacy of Anti-PSMA/Anti-CD3 Bispecific Antibodies in Human Tumor Xenograft Models

To assess the in vivo efficacy of the anti-PSMA/anti-CD3 bispecifics in human tumor xenograft studies, NOD scid gamma (NSG) mice (Jackson Laboratories, Bar Harbor, Maine) were co-implanted with human peripheral blood mononuclear cells (PBMCs) along with C4-2 human prostate tumor cells which endogenously express PSMA.

Briefly, 5×10⁶ C4-2 cells (MD Anderson, TX) cells were co-implanted s.c. with 1×10⁶ human PBMCs (ReachBio, LLC., Seattle, WA) in a 50:50 mix of matrigel matrix (BD Biosciences) into the right flank of male NSG mice. The mice were treated i.p. on days 0, 4, and 7 post tumor implantation with 0.1 mg/kg of PSMA/CD3-002.

In an additional xenogenic model, an anti-PSMA×anti-CD3 bispecific was tested in mice engrafted with human hematopoietic CD34+ stem cells. Briefly, newborn SIRPα BALB/c-Rag2-IL2rγ- (BRG) pups were engrafted with hCD34+ fetal liver cells. 3-6 months later hCD34-engrafted SIRPα BRG mice were then implanted with C4-2 cells (5×10⁶ s.c. in matrigel). 8 days later, mice were treated with 10 μg of PSMA/CD3-001 or an isotype control antibody, followed by 2×/week doses throughout the study.

In all studies, tumor size was measured 2×/week using calipers and tumor volume calculated as Volume=(length×width²)².

As the results in Table 6 show, the bispecific antibodies tested in the xenogenic models described above were both effective at inhibiting tumor growth compared to treatment with the isotype control.

### Efficacy of Anti-PSMA/Anti-CD3 Bispecific Antibodies in Immune-Competent Tumor Model

Additionally, an anti-PSMA×anti-CD3 bispecific was assessed for anti-tumor activity in an immune-competent model. Mice humanized for the three chains (Sys) of CD3 as well as for PSMA were implanted with a variant murine prostate cancer cell line TRAMP-C2 transfected with human PSMA.

Prior to study initiation, the tumorigenic cell line variant TRAMP-C2_hPSMAv #1 was generated. Briefly, 7.5×10⁶ TRAMP-C2_hPSMA cells were implanted s.c. into the right flank of male mice humanized for CD3 and PSMA. A tumor was excised and cut into 3 mm fragments and subsequently implanted into the right flank of new male humanized mice. A tumor arising from the implanted tumor fragments was then harvested and disaggregated into a single cell suspension. These cells (TRAMP-C2_hPSMAv #1) were then cultured in vitro under G418 selection. 4.10⁸ cells of this variant cell line were then implanted into the right flank of male PSMA/CD3 humanized mice for the bispecific antibody efficacy studies.

Humanized PSMA/CD3 mice implanted with TRAMPC2_hPSMAv #1 were treated with 100ug or 10ug of anti-PSMA×anti-CD3 bispecific antibody PSMA/CD3-001 or an isotype control 2×/week starting from the day of tumor implantation. Serum cytokine levels 4 h post-injection were also examined, as well as spleen T-cell levels. Study was terminated at Day 27.

As the results in Table 7 show, the anti-PSMA×anti-CD3 bispecific antibody showed efficacy in significantly delaying tumor growth across treatment groups. Minimal cytokine release was observed after administration of PSMA/CD3-001, possibly due to the weak binding of the anti-CD3.

Of note, in the absence of PSMA-expressing tumor cells, no T cell activation was seen.

Additionally, in mice bearing no tumors, blood samples were collected 4 hours following PSMAxCD3 bispecific antibody treatment, and serum cytokine levels were determined. Transient increases in levels of cytokines, namely interferon-gamma (IFN-g), tumor necrosis factor (TNF), interleukin-2 (IL-2), and interleukin-6 (IL-6) were determined and the transient increases were dose-dependent (data not shown).

TABLE 6
Efficacy of anti-PSMA/anti-CD3 Bispecific Antibodies in Immune-Compromised Xenograft Models
Xenogenic model: suppression of tumor growth

| | N | | | Final Tumor Volume |
|---|---|---|---|---|
| Tumor Model/ | # mice/ | Bispecific Antibody | | (mm³) |
| Mouse Strain | treatment group | Identifier | Dose | Mean ± SD |
| C4-/2 | 5 | PSMA/CD3-002 | 0.1 mg/kg | 0 ± 0 |
| NSG | 5 | Isotype Control | on day 0, 4 & 7 | 960 ± 660 |
| C4-2/ | 5 | PSMA/CD3-001 | 1.0 ug/mouse | 70 ± 60 |
| SIRPα Balb/c-Rag2- | 5 | Isotype Control | 2x/week | 260 ± 180 |
| IL2rγ- BRG engrafted | 4 | Isotype Control | 1170 ± 600 | (−) |
| with hCD34 + HSC | | | | |

TABLE 7
Efficacy of anti-PSMA/anti-CD3 Bispecific antibodies in immune-
competent syngeneic models (TRAMP-C2/PSMA^{Hum/hum}CD3^{Hum/Hum})

| | | N # | Tumor | Mean Serum Cytokine | | | | | Spleen T-cell | |
|---|---|---|---|---|---|---|---|---|---|---|
| Bispecific | Dose | mice/ | Volume (mm³) | Concentrations, (pg/mL) | | | | | level %, | |
| Antibody | (ug/mouse) | treatment | at Day 27 | | | | IL- | | (mean ± SD)^{#} | |
| Identifier | 2x/week* | group | (Mean ± SD) | IFNg | TNFa | IL-2 | 12p70 | IL-6 | CD4+ | CD8+ |
| PSMA/ | 100 | 4 | 50 ± 60 | 30 | 60 | 60 | 40 | 370 | 8.0 ± 1.0 | 12.0 ± 2.0 |
| CD3-001 | 10 | 5 | 380 ± 650 | 10 | 50 | 50 | 10 | 330 | 8.0 ± 3.0 | 14.0 ± 4.0 |
| Isotype | 100 | 5 | 1740 ± 560 | 4 | 30 | 30 | 10 | 230 | 5.0 ± 1.0 | 8.0 ± 2.0 |
| Control | | | | | | | | | | |

*Mice were dosed with antibody or isotype control 2x/week starting on the day of tumor implantation
^{#}Measured as the percentage of CD4+ or CD8+ cells in spleen out of live mCD45+ cells

### Example 6: A Phase 1/2 Study of a Bispecific Anti-PSMA×Anti-CD3 Antibody Administered Alone or in Combination with an Anti-PD-1 Antibody in Patients with Metastatic Castration-Resistant Prostate Cancer

This is an open-label, Phase 1/2, first-in-human, multicenter dose-escalation study with cohort expansion evaluating safety, tolerability, pharmacokinetics (PK), and antitumor activity of PSMA/CD3-002 administered subcutaneously alone and in combination with intravenous cemiplimab in patients with metastatic castration-resistant prostate cancer (mCRPC). Patients must have received at least two prior lines of systemic therapy approved for metastatic and/or castration-resistant disease including a second-generation anti-androgen therapy. PSMA/CD3-002 as monotherapy is administered weekly but may be extended to once every 3 weeks following identification of the minimal pharmacologically active dose. PSMA/CD3-002 in combination with cemiplimab (350 mg) will be administered once every 3 weeks after a 4-week PSMA/CD3-002 monotherapy lead-in cycle. Study therapies are administered until disease progression, intolerable adverse events, withdrawal of consent, or study withdrawal criterion is met.

The primary objectives in dose escalation are to evaluate the safety, tolerability, PK, and recommended phase 2 dosing regimen (RP2DR) of PSMA/CD3-002 alone and in-combination with cemiplimab. Expansion cohort(s) will be enrolled once RP2DRs have been determined. During the expansion phase, the primary objective is to assess clinical activity, as measured by objective response rate (ORR) with PSMA/CD3-002 alone or in combination with cemiplimab per modified Prostate Cancer Working Group 3 criteria. At selected sites, PSMA positron emission tomography/computed tomography scans will be performed at predefined timepoints on study. Additional details are provided below.

### Study Objectives

The primary objectives of the study are: to assess the safety, tolerability, and PK and to determine RP2DR of PSMA/CD3-002 separately as monotherapy or in combination with cemiplimab (in dose escalation); and to assess preliminary anti-tumor activity of PSMA/CD3-002 as monotherapy or in combination with cemiplimab as measured by objective response rate (ORR) per modified Prostate Cancer Working Group (PCWG3) criteria (in dose expansion).

The secondary objectives of the study are: to assess preliminary anti-tumor activity of PSMA/CD3-002 as monotherapy or in combination with cemiplimab as measured by ORR per modified PCWG3 criteria (in dose escalation); to characterize the safety profile in each expansion cohort (in does expansion); to characterize the PK of PSMA/CD3-002 as monotherapy or in combination with cemiplimab (in dose expansion); to assess preliminary anti-tumor activity of PSMA/CD3-002 as monotherapy or in combination with cemiplimab as measured by PSA decline (in dose escalation and dose expansion); and to evaluate immunogenicity of PSMA/CD3-002 in Module 1 and immunogenicity of PSMA/CD3-002 and cemiplimab in Module 2 (in dose escalation and dose expansion).

The exploratory objectives of the study are:
To assess preliminary anti-tumor activity of PSMA/CD3-002 as monotherapy or in combination with cemiplimab as measured by percent change in PSA, disease control rate (DCR), duration of response (DOR), radiographic progression-free survival (rPFS), PSA progression-free survival, overall survival (OS), time to response, and time to progression
To evaluate exploratory molecular biomarkers for the assessment of PSMA/CD3-002 impact, alone or in combination with cemiplimab, to understand PSMA/CD3-002 mechanism of action, observed toxicity, the disease/target, and genomic factors underlying disease and responsiveness in the patient population
To characterize the relationship between serum cytokine levels and clinical tolerability of PSMA/CD3-002 monotherapy or in combination with cemiplimab
To identify features of the tumor and its microenvironment in lesional tissue which are predictive of anti-tumor activity, or which respond pharmacodynamically to PSMA/CD3-002 therapy
To identify features of the peripheral immune cell population which are predictive of anti-tumor activity or toxicity, or which respond pharmacodynamically to PSMA/CD3-002 therapy
To evaluate the impact of PSMA/CD3-002 alone or in combination with cemiplimab on PSMA-positive and fluorodeoxyglucose (FDG)-positive tumors as assessed by PET/CT
To evaluate change from baseline in worst pain, average pain and pain interference in the daily activities scales of the Brief Pain Inventory—Short Form (BPI-SF)
To evaluate time to pain progression based on BPI-SF Item 3 “pain at its worst in the last 24 hours”
To evaluate the impact of PSMA/CD3-002 alone or in combination with cemiplimab on patient reported disease-specific symptoms, functioning and health-related quality of life measures (QLQ-C30, QLQ-PR25, PGIC, PGIS).
To evaluate the change in opioid medication usage in the patient population

### Study Design

This is a phase 1/2, first-in-human (FIH), open-label, multicenter study evaluating safety, tolerability, efficacy, and PK of PSMA/CD3-002, an anti-PSMA×anti-CD3 bispecific antibody (bsAb), administered subcutaneously (SC) as monotherapy (Module 1) or in combination with intravenously (IV) administered cemiplimab (Module 2) in patients with treatment-experienced metastatic castration-resistant prostate cancer (mCRPC). Module 1 will start first, Module 2 will begin after the minimum pharmacologically active dose level is identified in Module 1.

There are two parts of the study, dose escalation and dose expansion. All patients in the study will be treated with 2 successive “step-up” doses (initial dose and transitional dose) of PSMA/CD3-002 before reaching the target dose. The RP2DR, including the step-up dosing regimen and the target dose of PSMA/CD3-002, determined in dose escalation will be further evaluated in dose expansion.

Dose Escalation: Dose levels are defined by the target dose of PSMA/CD3-002. Five (or up to eight) potential dose levels are designed in which the target dose of PSMA/CD3-002 will be escalated in ½ (half)-log increments. Dose increments of <100% may be implemented based on observed toxicities at any given dose level.

Module 1—Determination of the step-up dosing regimen and dose escalation of monotherapy PSMA/CD3-002: The initial dose of the step-up dosing regimen starts at a dose of 0.03 mg SC. All three doses will be escalated in ½-log increments per dose level until the occurrence of specified adverse events (AEs) result in a modification to the dosing regimen (for initial and transitional doses) or the maximum tolerated dose (MTD)/RP2DR is defined (for the target dose). Administration of target doses will start on a once weekly (QW) schedule, and switch to once every 3 weeks (Q3W) after a minimum pharmacologically active dose has been reached as assessed by PSA or tumor response.

Module 2—Dose escalation of PSMA/CD3-002 in combination with cemiplimab: Patients enrolling in Module 2 will receive a 4-week PSMA/CD3-002 monotherapy lead-in cycle (Cycle 0; QW SC, including “step-up” doses). During Module 2, PSMA/CD3-002 will be administered Q3W SC with cemiplimab 350 mg Q3W IV (Cycle 1+). Dose escalation of PSMA/CD3-002 in Module 2 will begin at least 1 dose level below the minimum pharmacologically active dose level of PSMA/CD3-002 determined in Module 1.

Assessment of Dose-Limiting Toxicities in Dose Escalation: A dose limiting toxicity (DLT) is any adverse event (AE) that could preclude advancing to higher dose levels. The DLT criteria incorporate AEs reported with other bsAbs and checkpoint inhibitors. Toxicities will be graded according to the NCI-CTCAE v5.0, except for cytokine release syndrome (CRS) which will be graded according to current American Society for Transplantation and Cellular Therapy (ASTCT) criteria.

In both modules, dose level escalation (i.e., target dose escalation) will follow BOIN (Bayesian optimal interval) design rules applied with the following conditions: 1) DLTs associated with previously untested doses of PSMA/CD3-002 will be considered, 2) dose levels will be escalated until a MTD with a toxicity rate of 30% is observed for the target dose of PSMA/CD3-002, and 3) the maximum number of patients treated at a dose level is 12.

In Module 1, because the step-up dosing regimen consists of doses that has been tested and deemed tolerable as a target dose in prior escalation cohorts (after DL1 in Module 1), separate rules will be applied to modify the step-up dosing regimen based on the occurrence and severity of cytokine release syndrome (CRS) and other DLTs.

### DLT Observation Periods:

Module 1: The DLT observation period (minimum duration of 28 days) in Module 1 begins when the first dose of study drug is administered. Monitoring for DLTs continues for at least 2 weeks following the first administration of the PSMA/CD3-002 target dose at the given dose level. Due to the allowance of up to 2 weeks of dose delays during step-up dosing prior to the first administration of the target dose, the maximum duration of the DLT period is 42 days. DLTs observed during step-up dosing will be considered separately from DLTs observed at the target dose and be considered for modification of the step-up dosing regimen. DLTs observed at the target dose will determine tolerability of the target dose and considered for dose level escalation.

Module 2: The DLT observation period is defined as 21 days from the first dose of combination therapy (PSMA/CD3-002 and cemiplimab) beginning on cycle 1 day 1.

Dose Expansion: During dose expansion, patients will receive either monotherapy PSMA/CD3-002 (Module 1) or combination therapy with cemiplimab 350 mg IV Q3W (Module 2) at the assigned DL (e.g., RP2DR). If multiple DLs reveal pharmacodynamic activity and are well tolerated, up to 2 expansion cohorts in total may be opened. In both dose escalation and dose expansion, safety evaluations will be conducted at each study drug dosing visit. Radiographic response assessment will be performed every 9 weeks (Q9W) throughout the study. Investigators should continue treating patients with study drug until clinical or confirmed radiographic disease progression per modified PCWG3, intolerable AEs, elective discontinuation for clinical response, withdrawal of consent, or other study withdrawal criterion is met. Patients who do not withdraw consent will then be expected to continue off-treatment follow-up procedures.

### Study Duration

All patients will proceed through three successive periods of the trial: Screening, Treatment, and Follow-Up. The screening period is up to 28 days. The treatment period is divided into cycles to align with the frequency of tumor assessments. During the treatment period, treatment cycles are generally 9 weeks in both modules, with tumor assessments scheduled to occur at the beginning of each cycle. Module 2 includes 2 shorter cycles (cycles 0 and 1) that are described in detail below. Treatment will continue until either disease progression, intolerable AEs, elective discontinuation for clinical response, withdrawal of consent, or other study withdrawal criterion is met.

Module 1—Monotherapy PSMA/CD3-002: The Module 1 treatment period is comprised of 9-week cycles. During cycle 1, individual patients will receive at least 2 escalating, “step-up” doses (i.e., initial dose and transitional dose) of PSMA/CD3-002 before reaching the target dose. Administration of the first target dose of PSMA/CD3-002 may be delayed by up to 2 weeks to resolve symptoms of CRS during initial and/or transitional doses.

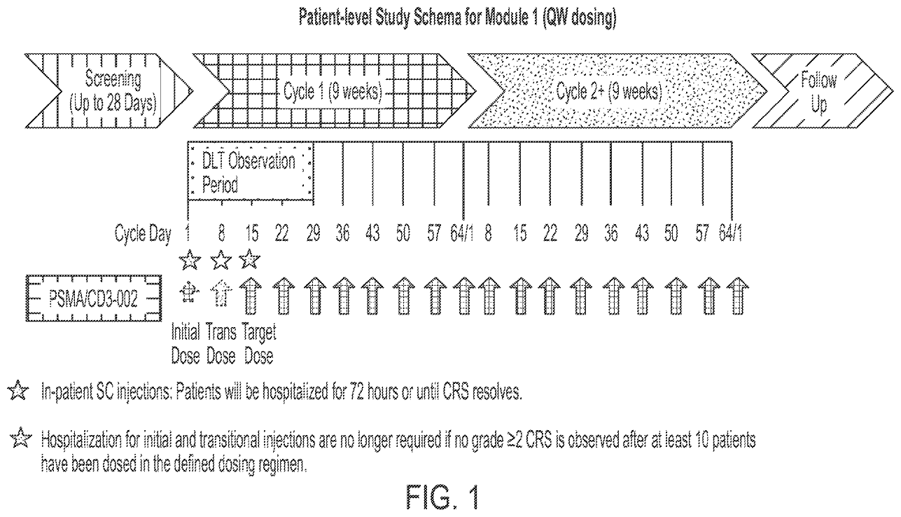

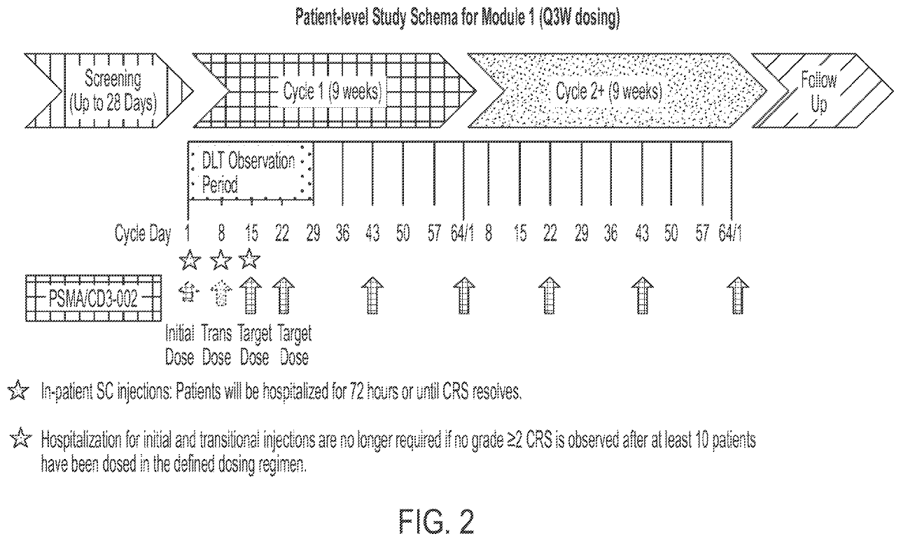

Patient-level study schema for Module 1 are provided in FIG. 1 (QW) and FIG. 2 (Q3W). As depicted in FIG. 1 and FIG. 2, during the first three administrations of PSMA/CD3-002, patients will be observed in a monitored setting for at least 72 hours or if CRS occurs, until systemic symptoms of CRS resolve. If grade ≥2 CRS is observed after administration, patients should continue to be observed in a monitored setting for each subsequent administration until post-injection CRS is grade ≤1, at which time their subsequent treatment administrations can occur in the outpatient setting.

After the minimum pharmacologically active dose level is identified, the next dose level will open with a Q3W schedule for the target dose of PSMA/CD3-002 for subsequently enrolled patients. The initial, transitional, and the first two consecutive target dosing intervals will remain QW for all patients enrolled in the study per FIG. 2. All doses will be administered SC.

For patients enrolled after the establishment of Q3W dosing and who require a lengthened step-up dosing period to resolve symptoms of CRS prior to receiving the first target dose, cycle 1 may be extended to maintain Q3W dosing schedule.

The dose escalation scheme and an alternative scheme for Module 1 are shown in Tables 8A and 8B3, below.

TABLE 8A
Dose Escalation Scheme for Module
1 (Monotherapy PSMA/CD3-002)

| DL¹ | Initial dose | Transitional dose | Target dose |
|---|---|---|---|
| DL1 | 0.03 mg | 0.1 mg | 0.3 mg |
| DL2 | 0.09 mg | 0.3 mg | 0.9 mg |
| DL3 | 0.3 mg | 0.9 mg | 3 mg |
| DL4 | 0.9 mg | 3 mg | 9 mg |
| DL5 | 3 mg | 9 mg | 30 mg |

¹The dosing schedule for the target dose of PSMA/CD3-002 may be QW or Q3W.

TABLE 8B
Alternative Dose Escalation Scheme for
Module 1 (Monotherapy PSMA/CD3-002)

| DL¹ | Initial dose | Transitional dose | Target dose |
|---|---|---|---|
| DL1 | 0.03 mg | 0.1 mg | 0.3 mg |
| DL2 | 0.1 mg | 0.3 mg | 1 mg |
| DL3 | 0.3 mg | 1 mg | 3 mg |
| DL4 | 1 mg | 3 mg | 10 mg |
| DL5 | 3 mg | 10 mg | 30 mg |
| DL6 | 10 mg | 30 mg | 100 mg |
| DL7 | 30 mg | 100 mg | 300 mg |
| DL8 | 100 mg | 300 mg | 900 mg |

¹The dosing schedule for the target dose of PSMA/CD3-002 may be QW or Q3W.

Module 2—Combination Therapy with PSMA/CD3-002 and Cemiplimab: The treatment period for patients enrolled in Module 2 will begin with a PSMA/CD3-002 monotherapy lead-in cycle (cycle 0), comprising of weekly step-up dosing. Due to the possible potentiation of CRS with combination therapy, patients must tolerate the target dose of PSMA/CD3-002 without CRS during the monotherapy lead-in prior to initiating combination with cemiplimab.

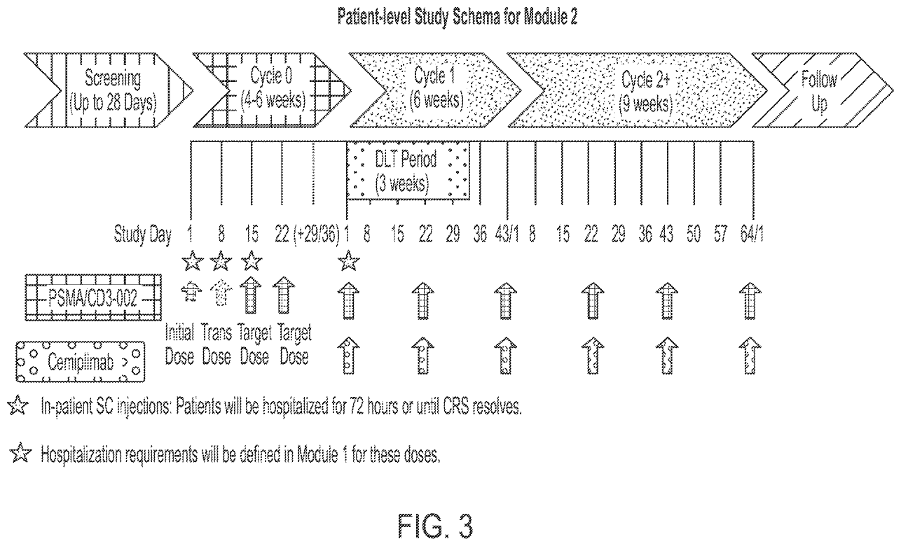

Dosing will be QW during cycle 0 and will transition to Q3W at cycle 1. Cycle 0 consists of weekly monotherapy dosing of PSMA/CD3-002 as initial, transitional, and 2 target doses. The planned length of cycle 0 is 4 weeks but may be extended up to 6 weeks to allow for the resolution of CRS from initial and transitional doses or to repeat the target dose to demonstrate tolerability without symptoms of CRS. Cycle 1, when PSMA/CD3-002 and cemiplimab combination therapy is initiated, is 6 weeks (42 days) to accommodate the timing of the first on-treatment tumor assessment. Subsequent cycles (cycles 2+) will be 9 weeks (63 days). Patient-level study schema for Module 2 is provided in FIG. 3.

In Module 2, the target dose of PSMA/CD3-002 will not exceed the target dose of PSMA/CD3-002 that has been deemed tolerable in Module 1. All PSMA/CD3-002 doses will be administered SC, and cemiplimab will be administered by IV. Patients will be observed in a monitored setting for the first three injections in cycle 0 if grade 2 CRS was observed in Module 1 for the same doses (FIG. 5) All patients will be observed in a monitored setting for the first combination treatment in cycle 1 for at least 72 hours or until all symptoms of CRS resolve. In addition, if grade ≥2 CRS is observed upon initial combination administration, then patients should continue to receive treatment in a monitored setting for each subsequent administration until post-injection CRS is grade ≤1, at which time their subsequent treatment can be administered as an outpatient.

TABLE 9A
Dose Escalation Scheme for Module 2 (PSMA/CD3-
002 in Combination with Cemiplimab)

| DL^{1,2} | Initial dose ^{QW} | Transitional dose ^{QW} | Target dose^{Q3W} | Cemiplimab^{Q3W} |
|---|---|---|---|---|
| DL1 | 0.03 | 0.1 | 0.3 mg | 350 mg |
| DL2 | derived from M1 | derived from M1 | 0.9 mg | 350 mg |
| DL3 | derived from M1 | derived from M1 | 3 mg | 350 mg |
| DL4 | derived from M1 | derived from M1 | 9 mg | 350 mg |
| DL5 | derived from M1 | derived from M1 | 30 mg | 350 mg |

¹If Module 2 begins at a dose level in which the initial and transitional doses are defined in Module 1, those defined doses will be implemented in Module 2.
²If Module 2 begins prior to the definition of initial and transitional doses, then the same doses of PSMA/CD3002 as specified in Table 8A will be used for initial and transitional doses in Module 2.

TABLE 9B
Alternative Dose Escalation Scheme for Module
2 (PSMA/CD3-002 in Combination with Cemiplimab)

| DL^{1,2} | Initial dose ^{QW} | Transitional dose ^{QW} | Target dose^{Q3W} | Cemiplimab^{Q3W} |
|---|---|---|---|---|
| DL1 | 0.03 | 0.1 | 0.3 mg | 350 mg |
| DL2 | derived from M1 | derived from M1 | 1 mg | 350 mg |
| DL3 | derived from M1 | derived from M1 | 3 mg | 350 mg |
| DL4 | derived from M1 | derived from M1 | 10 mg | 350 mg |
| DL5 | derived from M1 | derived from M1 | 30 mg | 350 mg |
| DL6 | derived from M1 | derived from M1 | 100 mg | 350 mg |
| DL7 | derived from M1 | derived from M1 | 300 mg | 350 mg |
| DL8 | derived from M1 | derived from M1 | 900 mg | 350 mg |

¹If Module 2 begins at a dose level in which the initial and transitional doses are defined in Module 1, those defined doses will be implemented in Module 2.
²If Module 2 begins prior to the definition of initial and transitional doses, then the same doses of PSMA/CD3-002 as specified in Table 8B will be used for initial and transitional doses in Module 2.

### Study Population

Approximately 199 patients will be enrolled for the study, as follows: Anticipated enrollment is approximately 91 patients in the dose escalation phase (43 patients in Module 1 and 48 patients in Module 2); and Approximately 108 patients in the dose expansion phase. Sample size estimation is based on up to 2 expansion cohorts for each module with up to 27 patients per cohort.

The study population includes men with treatment-experienced mCRPC. For inclusion in this study, patients must have received at least 2 approved therapies for metastatic and/or castration-resistant disease, including a second-generation anti-androgen therapy (e.g., abiraterone, enzalutamide, apalutamide, or darolutamide).

Inclusion Criteria: A patient must meet the following criteria to be eligible for inclusion in the study:
1. Males ≥18 years of age
2. Histologically or cytologically confirmed adenocarcinoma of the prostate without pure small cell carcinoma
3. Metastatic, castration-resistant prostate cancer (mCRPC) with PSA value at screening ≥4 ng/mL that has progressed within 6 months prior to screening according to 1 of the following:
   a. PSA progression as defined by a rising PSA level confirmed with an interval of ≥1 week between each assessment.
   b. Radiographic disease progression in soft tissue based on RECIST Version 1.1 criteria with or without PSA progression
   c. Radiographic disease progression in bone defined as the appearance of 2 or more new bone lesions on bone scan with or without PSA progression. NOTE: Measurable lesion per RECIST Version 1.1 per local reading at screening is not an eligibility criterion for enrollment
4. Has progressed upon or is intolerant to ≥2 lines prior systemic therapy approved in the metastatic and/or castration resistant setting (in addition to androgen deprivation therapy [ADT]) including at least 1 second-generation anti-androgen therapy (eg, abiraterone, enzalutamide, apalutamide, or darolutamide) NOTE: a non-taxane based chemotherapy regimen given for metastatic prostate cancer with mixed histology is permissible and will be included when evaluating line of therapy
5. Able and willing to provide tumor tissue, either archival or newly obtained. NOTE: For dose escalation only, if archival or fresh tissue is not available, a pathology report that confirms diagnosis of prostate cancer may be submitted
6. Have had either orchiectomy OR be on luteinizing hormone-releasing hormone (LHRH) agonist or antagonist therapy with serum testosterone <50 ng/dL AND agree to stay on LHRH agonist or antagonist therapy during the study
7. Has an ECOG performance status of 0 or 1
8. Has adequate organ and bone marrow function documented by:
   a. Hemoglobin ≥8.5 g/dL
   b. Absolute neutrophil count ≥1.0×10⁹/L
   c. Platelet count ≥100×10⁹/L
9. Serum creatinine ≤1.5×ULN or estimated glomerular filtration rate >50 mL/min/1.73 m². A 24-hour urine creatinine collection may substitute for the calculated creatinine clearance to meet eligibility criteria.
10. Adequate hepatic function:
   (1) Total bilirubin ≤1.5×ULN; for patients with known Gilbert's syndrome, ≤3× the institutional ULN is permitted
   (2) AST ≤2.5×ULN
   (3) ALT ≤2.5×ULN
   (4) ALP ≤2.5×ULN
11. Is willing and able to comply with clinic visits and study-related procedures and requirements
12. Is willing and able to provide informed consent as specified by health authorities and institutional guidelines
13. Is able to understand and complete study-related questionnaires

Exclusion Criteria: A patient who meets any of the following criteria will be excluded from the study:
1. Currently receiving treatment in another study
2. Has participated in a study of an investigational agent or an investigational device within 4 weeks of first dose of study therapy
3. Has received treatment with an approved systemic therapy (including sipuleucel-T) within 3 weeks of dosing or has not yet recovered (i.e., grade ≤1 or baseline) from any acute toxicities except for laboratory changes as described in inclusion criteria and as below:
   a. Patients with grade ≤2 neuropathy
4. Has received radiation therapy or major surgery within 14 days of first administration of study drug or has not recovered (ie, grade ≤1 or baseline) from AEs, except for laboratory changes as described in inclusion criteria and as below:
   a. Patients with grade ≤2 neuropathy
5. Has received any previous systemic biologic therapy within 5 half-lives of first dose of study therapy. Note: Patients previously treated with cetuximab, rituximab, or other non-immunomodulatory antibodies with half-lives longer than 7 days are permitted after a discussion with the sponsor if at least 30 days have elapsed since last treatment
6. Has received prior PSMA-targeting therapy
7. Dose Escalation and Module 1 Dose Expansion: Has had prior anti-cancer immunotherapy (other than sipuleucel-T) within 5 half-lives prior to study therapy. Examples of immune modulating agents include blockers of CTLA-4, 4-1BB (CD137), or OX-40, therapeutic vaccines, anti-PD-1/PD-L1, phosphoinositide 3-kinase (PI3K) delta inhibitors, or cytokine anticancer treatments. NOTE: Patients who have received prior investigational cell-based therapies (e.g., CAR-T cells) are excluded
8. Module 2 Dose Expansion: Has had prior anti-cancer immunotherapy. Examples of immune modulating agents include blockers of CTLA-4, 4-1BB (CD137), or OX-40, therapeutic vaccines, anti-PD-1/PD-L1, PI3Kdelta inhibitors, CAR-T cells, or cytokine anticancer treatments. NOTE: Prior treatment with sipuleucel-T is permitted
9. Patients who have not recovered (i.e., grade ≤1 or baseline) from immune-mediated AEs 3 months prior to initiation of study drug therapy except for endocrinopathies adequately managed with hormone replacement
10. Patients who have permanently discontinued anti-cancer immune modulating therapies due to immune-related AEs
11. Has any condition requiring ongoing/continuous corticosteroid therapy (>10 mg prednisone/day or anti-inflammatory equivalent) within 1 week prior to the first dose of study drug. Physiologic replacement doses are allowed even if they are >10 mg of prednisone/day or equivalent, as long as they are not being administered for immunosuppressive intent. Inhaled or topical steroids are permitted, provided that they are not for treatment of an autoimmune disorder. Note: Patients who require a brief course of steroids (up to 2 days in the week before enrollment) or physiologic replacement may be enrolled into the study
12. Has ongoing or recent (within 5 years) evidence of significant autoimmune disease that required treatment with systemic immunosuppressive treatments. The following are not exclusionary: vitiligo, childhood asthma that has resolved, endocrinopathies (such as hypothyroidism or type 1 diabetes) that require only hormone replacement, or psoriasis that does not require systemic treatment.
13. Has liver metastases
14. Has another malignancy that is progressing or requires active treatment, except:
   a. Non-melanoma skin cancer that has undergone potentially curative therapy
   b. Any tumor that has been deemed to be effectively treated with definitive local control (with or without continued adjuvant hormonal therapy)
15. Dose Escalation: History of CNS metastases, including previously treated metastases Dose Expansion: Untreated or active primary brain tumor, CNS metastases, leptomeningeal disease, or spinal cord compression Exception: Patients with previously treated central nervous system metastases or spinal cord compression may participate provided there is:
   a. No evidence of progression for at least 6 weeks prior to the first dose of study drug, and any neurologic symptoms have returned to baseline
   b. No evidence of new or enlarging central nervous system metastases
   c. No requirement for systemic corticosteroids for management of central nervous system metastases or spinal cord compression within 2 weeks prior to the first dose of study drug
16. Has encephalitis, meningitis, neurodegenerative disease (with the exception of mild dementia that does not interfere with activities of daily living [ADLs]) or uncontrolled seizures in the year prior to first dose of study therapy
17. Has a known history of, or any evidence of, interstitial lung disease or active, noninfectious pneumonitis within 5 years prior to the first dose of study drug. A history of radiation pneumonitis in the radiation field is permitted
18. Has uncontrolled infection with human immunodeficiency virus, hepatitis B, or hepatitis C infection; or has a diagnosis of immunodeficiency
   a. Patients will be tested for hepatitis C virus (HCV) and hepatitis B virus (HBV) at screening
   b. Patients with known HIV infection who have controlled infection (undetectable viral load (HIV RNA PCR) and CD4 count above 350 either spontaneously or on a stable antiviral regimen) are permitted. For patients with controlled HIV infection, monitoring will be performed per local standards
   c. Patients with hepatitis B (HBsAg+) who have controlled infection (serum hepatitis B virus DNA PCR that is below the limit of detection and receiving anti-viral therapy for hepatitis B) are permitted. Patients with controlled infections must undergo periodic monitoring of HBV DNA. Patients must remain on anti-viral therapy for at least 6 months beyond the last dose of investigational study drug
   d. Patients who are hepatitis C virus antibody positive (HCV Ab+) who have controlled infection (undetectable HCV RNA by PCR either spontaneously or in response to a successful prior course of anti-HCV therapy) may be enrolled into the study
19. Has any infection requiring hospitalization or treatment with intravenous anti-infectives within 2 weeks of first dose of study drug
20. Has received a live vaccine within 28 days of planned start of study drug
21. Has had prior allogeneic stem cell transplantation or received organ transplants at any time, or autologous stem cell transplantation within 12 weeks of the start of study drug
22. Has known allergy or hypersensitivity to components of study drugs
23. Has known psychiatric or substance abuse disorders that would interfere with participation with the requirements of the study
24. Has any medical condition, co-morbidity, physical examination finding, or metabolic dysfunction, or clinical laboratory abnormality that, in the opinion of the investigator, renders the patient unsuitable for participation in a clinical study due to high safety risks and/or potential to affect interpretation of results of the study including, but not limited to, significant cardiovascular disease (eg, New York Heart Association Class III or IV cardiac disease, myocardial infarction within the previous 6 months, unstable arrhythmias or unstable angina) and/or significant pulmonary disease (eg, obstructive pulmonary disease and history of symptomatic bronchospasm)
25. Has a cardiac ejection fraction <40% by echocardiogram or multi-gated acquisition scan (MUGA)

### Study Treatments

PSMA/CD3-002 will be administered at a dose of from 0.3 mg to 30 mg (but may be up to 900 mg) either QW or Q3W by SC administration as target dose levels.

Cemiplimab will be administered at a dose of 350 mg concomitantly Q3W by IV infusion over 30 minutes.

When both drugs are administered on the same day, PSMA/CD3-002 will be administered first and cemiplimab administration should follow no sooner than 30 minutes following completion of PSMA/CD3-002 administration.

Routine premedication with steroids will be required for all enrolled patients starting at C1D1, as detailed below:
10 mg dexamethasone IV/PO on the day of and day after dosing through 2^{nd} full dose (e.g., C1D22)
Initiate taper after second full dose
   10 mg IV/PO on 3^{rd} full dose (e.g., C1D29)
   6 mg IV/PO on 4^{th} full dose (e.g., C1D36)

### Study Endpoints

The primary endpoints of the study are: Dose-limiting toxicities, other treatment-emergent adverse events (TEAEs; including irAEs), serious AEs (SAEs), adverse events of special interests (AESIs), and laboratory abnormalities (in dose escalation); PSMA/CD3-002 concentrations in serum as monotherapy or in combination with cemiplimab (in dose escalation); and Objective Response Rate (ORR) per modified Prostate Cancer Working Group 3 (PCWG3) criteria, (in dose expansion) defined as the percentage of patients who have achieved response based on:
≥50% decline of PSA from baseline, confirmed by a second PSA test ≥4 weeks later, AND/OR
Confirmed radiographic response of complete response (CR) or partial response (PR)

The key secondary endpoints are: ORR per modified PCWG3 criteria (in dose escalation), defined as the percentage of patients who have achieved response based on ≥50% decline of PSA from baseline, confirmed by a second PSA test 4 weeks later, and/or confirmed radiographic response of complete response (CR) or partial response (PR); dose-limiting toxicities, other TEAEs (including irAEs), SAEs, AESIs, and laboratory abnormalities (in does expansion); PSMA/CD3-002 concentrations in serum as monotherapy or in combination with cemiplimab (in dose expansion); percentage of patients with ≥50% reduction PSA from baseline, confirmed by a second PSA test ≥4 weeks later (in dose escalation and expansion); percentage of patients with ≥90% reduction PSA from baseline, confirmed by a second PSA test 4 weeks later (in dose escalation and expansion); and immunogenicity, as measured by anti-drug antibodies (ADA) to PSMA/CD3-002 in Module 1 and ADA to PSMA/CD3-002 and cemiplimab in Module 2 (in dose escalation and expansion).

The exploratory endpoints are:
Percent change in PSA
Disease Control Rate (DCR) (via modified PCWG3 and iRECIST)
Duration of Response (DOR), based upon radiographic response (rDOR) (via modified PCWG3 and iRECIST)
DOR based upon PSA response
Radiographic Progression-free Survival (rPFS) (via modified PCWG3 and iRECIST)
PSA progression free survival
Overall survival (OS)
Time to response based upon radiographic response (via modified PCWG3 and iRECIST)
Time to response based upon PSA response
Time to progression based upon radiographic progression
Time to progression based upon PSA progression
Abundance and distribution of therapeutic targets (e.g., PD-1/PD-L1, PSMA, and CD3+ T cells) in tumor tissue samples at baseline and over time on therapy
Association of CTC abundance and molecular features with clinical efficacy
Tumor mutational profiling of ctDNA, CTCs, and/or tumor tissue at baseline and on treatment, including assessment of individual somatic variants, tumor mutational burden (TMB), alterations of the DNA repair pathway genes, and evaluation of the relationship between these features and drug response
Abundance and phenotype of circulating T-cell subsets at baseline and on treatment, and the association of their pharmacodynamic responsiveness with clinical efficacy or toxicity
Concentrations of systemic inflammatory markers (e.g., serum cytokines) and immune activity and clinical toxicity
Changes from baseline in tumor PSMA and FDG PET signal after therapy initiation
Association of baseline PSMA PET tumor positivity with clinical activity
Time to Pain Progression (TTPP) as Assessed by Brief Pain Inventory-Short Form (BPI-SF) Item 3 (“Worst Pain in 24 Hours”) and Opiate Analgesic Use
Change from baseline in pain severity and pain interference as measured by the BPI-SF
Change from baseline in GHS/QoL as measured by the EORTC QLQ-C30 GHS/QoL scale score
Change from baseline in Physical functioning as measured by the EORTC QLQ-C30 physical functioning scale score
Change from baseline in urinary symptoms as measured by the EORTC QLQ-PR25 urinary symptom scale score

Results: It is expected that administration of PSMA/CD3-002 and cemiplimab in patients with prostate cancer will result in complete response or partial response with durable disease control.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 10
Sequences Excluded from ST.26-Formatted Sequence Listing

| SEQ ID NO: | Sequence |
|---|---|
| 9 | AAS |
| 27 | AAS |

What is claimed is:

1. A method of treating a PSMA-expressing cancer in a subject in need thereof, comprising administering to the subject a bispecific antibody comprising a first antigen-binding domain that specifically binds prostate specific membrane antigen (PSMA) on a target tumor cell, and a second antigen-binding domain that specifically binds human CD3 on a T cell, wherein the bispecific antibody is administered to the subject at a dose of at least 0.03 mg.

2. The method of claim 1, wherein the PSMA-expressing cancer is prostate cancer.

3. The method of claim 1 or 2, wherein the PSMA-expressing cancer is metastatic prostate cancer.

4. The method of any one of claims 1-3, wherein the PSMA-expressing cancer is castration-resistant prostate cancer.

5. The method of any one of claims 1-4, wherein the subject has received at least two prior therapies for metastatic and/or castration-resistant prostate cancer.

6. The method of claim 5, wherein the subject has received at least one anti-androgen therapy.

7. The method of claim 6, wherein the anti-androgen therapy is selected from abiraterone, enzalutamide, apalutamide, or darolutamide.

8. The method of any one of claims 1-7, wherein the subject has histologically or cytologically confirmed adenocarcinoma of the prostate without pure small cell carcinoma.

9. The method of any one of claims 1-8, wherein the subject has metastatic castration-resistant prostate cancer with a prostate specific antigen (PSA) value of ≥4 ng/ml prior to treatment with the bispecific antibody.

10. The method of claim 9, wherein the subject's cancer has progressed within a six month period prior to treatment with the bispecific antibody, wherein cancer progression is determined by: (a) a rising PSA level confirmed with an interval of ≥1 week between each assessment; (b) radiographic disease progression in soft tissue with or without a rise in PSA; and/or (c) radiographic disease progression in bone with an appearance of two or more bone lesions on bone scan with or without a rise in PSA.

11. The method of any one of claims 1-10, wherein the subject has had an orchiectomy.

12. The method of any one of claims 1-10, wherein the subject is receiving luteinizing hormone-releasing hormone (LHRH) agonist or antagonist therapy, and has a serum testosterone level of <50 ng/ml prior to treatment with the bispecific antibody.

13. The method of any one of claims 1-12, wherein the first antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 1; and
(b) three light chain complementarity determining regions (LCDR1, LCDR2 and LCDR3) contained within a light chain variable region (LCVR) comprising the amino acid sequence of SEQ ID NO: 2.

14. The method of claim 13, wherein the first antigen-binding domain comprises a HCDR1 comprising the amino acid sequence of SEQ ID NO: 5, a HCDR2 comprising the amino acid sequence of SEQ ID NO: 6, and a HCDR3 comprising the amino acid sequence of SEQ ID NO: 7.

15. The method of claim 13 or 14, wherein the first antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10.

16. The method of any one of claims 13-15, wherein the first antigen-binding domain comprises a HCVR comprising the amino acid sequence of SEQ ID NO: 1, and a LCVR comprising the amino acid sequence of SEQ ID NO: 2.

17. The method of any one of claims 1-16, wherein the second antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 4; and
(b) three light chain complementarity determining regions (LCDR1, LCDR2 and LCDR3) contained within a light chain variable region (LCVR) comprising the amino acid sequence of SEQ ID NO: 2.

18. The method of claim 17, wherein the second antigen-binding domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 16.

19. The method of claim 17 or 18, wherein the second antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10.

20. The method of any one of claims 17-19, wherein the second antigen-binding domain comprises a HCVR comprising the amino acid sequence of SEQ ID NO: 4, and a LCVR comprising the amino acid sequence of SEQ ID NO: 2.

21. The method of any one of claims 1-16, wherein the second antigen-binding domain comprises:
(a) three heavy chain complementarity determining regions (HCDR1, HCDR2 and HCDR3) contained within a heavy chain variable region (HCVR) comprising the amino acid sequence of SEQ ID NO: 3; and
(b) three light chain complementarity determining regions (LCDR1, LCDR2 and LCDR3) contained within a light chain variable region (LCVR) comprising the amino acid sequence of SEQ ID NO: 2.

22. The method of claim 21, wherein the second antigen-binding domain comprises a HCDR1 comprising the amino acid sequence of SEQ ID NO: 11, a HCDR2 comprising the amino acid sequence of SEQ ID NO: 12, and a HCDR3 comprising the amino acid sequence of SEQ ID NO: 13.

23. The method of claim 21 or 22, wherein the second antigen-binding domain comprises a LCDR1 comprising the amino acid sequence of SEQ ID NO: 8, a LCDR2 comprising the amino acid sequence of SEQ ID NO: 9, and a LCDR3 comprising the amino acid sequence of SEQ ID NO: 10.

24. The method of any one of claims 21-23, wherein the second antigen-binding domain comprises a HCVR comprising the amino acid sequence of SEQ ID NO: 3, and a LCVR comprising the amino acid sequence of SEQ ID NO: 2.

25. The method of any one of claims 1-24, wherein the bispecific antibody comprises a human IgG heavy chain constant region.

26. The method of claim 25, wherein the human IgG heavy chain constant region is isotype IgG1.

27. The method of claim 25, wherein the human IgG heavy chain constant region is isotype IgG4.

28. The method of claim 26 or 27, wherein the bispecific antibody comprises a chimeric hinge that reduces Fcγ receptor binding relative to a wild-type hinge of the same isotype.

29. The method of any one of claims 25-28, wherein the first heavy chain or the second heavy chain, but not both, comprises a CH3 domain comprising a H435R (EU numbering) modification and a Y436F (EU numbering) modification.

30. The method of any one of claims 1-24, wherein the bispecific antibody comprises a first heavy chain comprising the amino acid sequence of SEQ ID NO: 17.

31. The method of any one of claims 1-24, wherein the bispecific antibody comprises a second heavy chain comprising the amino acid sequence of SEQ ID NO: 20.

32. The method of any one of claims 1-24, wherein the bispecific antibody comprises a second heavy chain comprising the amino acid sequence of SEQ ID NO: 19.

33. The method of any one of claims 1-24, wherein the bispecific antibody comprises a first heavy chain comprising the amino acid sequence of SEQ ID NO: 17, a second heavy chain comprising the amino acid sequence of SEQ ID NO: 20, and a common light chain comprising the amino acid sequence of SEQ ID NO: 18.

34. The method of any one of claims 1-24, wherein the bispecific antibody comprises a first heavy chain comprising the amino acid sequence of SEQ ID NO: 17, a second heavy chain comprising the amino acid sequence of SEQ ID NO: 19, and a common light chain comprising the amino acid sequence of SEQ ID NO: 18.

35. The method of any one of claims 1-34, further comprising administering a second therapeutic agent or therapeutic regimen.

36. The method of claim 35, wherein the second therapeutic agent or therapeutic regimen comprises an anti-PD-1 antibody or antigen-binding fragment thereof.

37. The method of claim 36, wherein the anti-PD-1 antibody or antigen-binding fragment comprises:
(a) three heavy chain complementarity determining regions (HCDR1, HCDR2 and HCDR3) contained within a heavy chain variable region (HCVR) comprising the amino acid sequence of SEQ ID NO: 21; and
(b) three light chain complementarity determining regions (LCDR1, LCDR2 and LCDR3) contained within a light chain variable region (LCVR) comprising the amino acid sequence of SEQ ID NO: 22.

38. The method of claim 37, wherein the anti-PD-1 antibody or antigen-binding fragment comprises a HCDR1 comprising the amino acid sequence of SEQ ID NO: 23, a HCDR2 comprising the amino acid sequence of SEQ ID NO: 24, and a HCDR3 comprising the amino acid sequence of SEQ ID NO: 25.

39. The method of claim 37 or 38, wherein the anti-PD-1 antibody or antigen-binding fragment comprises a LCDR1 comprising the amino acid sequence of SEQ ID NO: 26, a LCDR2 comprising the amino acid sequence of SEQ ID NO: 27, and a LCDR3 comprising the amino acid sequence of SEQ ID NO: 28.

40. The method of any one of claims 37-39, wherein the anti-PD-1 antibody or antigen-binding fragment comprises a HCVR comprising the amino acid sequence of SEQ ID NO: 21, and a LCVR comprising the amino acid sequence of SEQ ID NO: 22.

41. The method of claim 40, wherein the anti-PD-1 antibody or antigen-binding fragment is an anti-PD-1 antibody comprising a heavy chain comprising the amino acid sequence of SEQ ID NO: 29 and a light chain comprising the amino acid sequence of SEQ ID NO: 30.

42. The method of any one of claims 1-41, wherein the bispecific antibody is administered to the subject at a dose of from 0.01 mg to 1000 mg weekly.

43. The method of claim 42, wherein the bispecific antibody is administered to the subject at a dose of from 0.03 mg to 30 mg weekly.

44. The method of claim 42, wherein the bispecific antibody is administered to the subject at a dose of from 3 mg to 900 mg weekly.

45. The method of any one of claims 1-41, wherein the bispecific antibody is administered to the subject at a dose of from 0.01 mg to 1000 mg once every three weeks.

46. The method of claim 45, wherein the bispecific antibody is administered to the subject at a dose of from 0.03 mg to 30 mg once every three weeks.

47. The method of claim 45, wherein the bispecific antibody is administered to the subject at a dose of from 3 mg to 900 mg once every three weeks.

48. The method of any one of claims 36-47, wherein the anti-PD-1 antibody is administered to the subject at a dose of from 300 to 400 mg once every three weeks.

49. The method of claim 48, wherein the anti-PD-1 antibody is administered to the subject at a dose of 350 mg once every three weeks.

50. The method of any one of claims 1-49, wherein the subject has stable disease, a partial response, or a complete response following administration of the bispecific antibody for at least one week at a dose of from 0.03 mg to 900 mg.

51. The method of any one of claims 1-50, wherein the subject is subjected to radiographic imaging following administration of one or more doses of the bispecific antibody.

52. The method of claim 51, wherein the radiographic imaging comprises a Fluorine F18 DCFPyL PET/CT scan.
